(19) United States
(12) Patent Application Publication
(10) Pub. No.: US 2014/0188159 A1
(43) Pub. Date: Jul. 3, 2014

(54) SURGICAL TOOL

(71) Applicant: AGILE ENDOSURGERY, INC., Chapel Hill, NC (US)

(72) Inventor: Adam T. C. Steege, Chapel Hill, NC (US)

(73) Assignee: AGILE ENDOSURGERY, INC., Chapel Hill, NC (US)

(21) Appl. No.: 14/232,123

(22) PCT Filed: Jul. 9, 2012
(86) PCT No.: PCT/US12/45951

§ 371 Date: Feb. 28, 2014

Related U.S. Application Data
(60) Provisional application No. 61/506,454, filed on Jul. 11, 2011.
(60) Provisional application No. 61/506,448, filed on Jul. 11, 2011.

(51) Int. Cl. A61B 17/28

(52) U.S. Cl. CPC A61B 17/282
USPC 606/207

(57) ABSTRACT

A surgical tool for minimally invasive surgery. In one embodiment, the surgical tool includes a proximal joint and a distal joint at opposite ends of a tube that constrain pivoting of adjacent parts to be about two perpendicular, intersecting axes. Three articulation control cable lengths engage and operatively couple the joints to control the two degrees of freedom of the distal joint. A manipulator and an end effector may be operatively coupled, with one cable controlling the opening and closing of jaws. In another embodiment, a surgical tool includes two flexible articulation elements mounted to opposite ends of a tube assembly. The tube assembly includes a proximal tube element and a distal tube element, with their ends rotatably mounted to each other. The mode of operation the tool may be changed between motion-following and motion-mirroring by rotating the distal tube element relative to the proximal tube element.

### CROSS-REFERENCE TO RELATED APPLICATIONS

This application claims the benefit of priority of U.S. Provisional Application No. 61/506,448, filed Jul. 11, 2011, entitled “Surgical Tool,” and U.S. Provisional Application No. 61/506,454, filed Jul. 11, 2011, entitled “Surgical Tool,” the contents of both of which are hereby incorporated by reference in their entirety.

### FIELD

Aspects of the present disclosure generally relate to surgical apparatus for tissue and suture manipulation, and more particularly may relate to apparatus that may be applied to conducting laparoscopic and endoscopic surgery.

### BACKGROUND

Minimally invasive surgery, such as endoscopic surgery, encompasses a set of techniques and tools which are becoming more and more commonplace in the modern operating room. Minimally invasive surgery causes less trauma to the patient when compared to the equivalent invasive procedure. Hospitalization time, scarring, and pain are also decreased, while recovery rate is increased.

Endoscopic surgery is accomplished by the insertion of a cannula containing a trocar to allow passage of endoscopic tools. Optics for imaging the interior of the patient, as well as fiber optics for illumination and an array of grasping and cutting devices are inserted through a multiple cannulae, each with its own port.

Currently the majority of cutting and grasping tools are essentially the same in their basic structure. Standard devices include a user interface at the proximal end and an end effector at the distal end of the tool used to manipulate tissue and sutures. Connecting these two ends is a tube section, containing cables and/or rods used for transmitting motion from the user interface at the proximal end of the tool to the end effector at the distal end of the tool. The standard minimally invasive devices (MIDs) provide limited freedom of movement to the surgeon. The cannula has some flexibility of movement at the tissue wall, and the tool can rotate within the cannula, but tools cannot articulate within the patient's body, limiting their ability to reach around or behind organs or other large objects. Several manually operated devices have attempted to solve this problem with articulated surgical tools that are controlled much in the same way as standard MIDs. These devices may have convoluted interfaces, making them more difficult to control than their robotic counterparts. Many lack torsional rigidity, limiting their ability to manipulate sutures and denser tissue.

Robotic surgical instruments have attempted to solve the problems that arise from the limitations of standard MIDs with telemetrically controlled articulated surgical tools. However, these tools are often prohibitively expensive to purchase and operate. The complexity of the devices may raise the cost of purchasing as well as the cost of a service contract. These robotic solutions also may have several other disadvantages such as complications during the suturing process. An additional disadvantage can be difficulty in providing haptic feedback.

Current devices may have excessive complexity of design and performance shortcomings. They may also lack the ability to be adaptive to the preferences of the surgeon.

### SUMMARY

In accordance with one embodiment, a surgical tool for use by an operator is provided. The surgical tool includes a proximal joint that constrains pivoting of parts that are mounted and adjacent to the proximal joint to be about two perpendicular, intersecting axes including a primary axis and a secondary axis such that the proximal joint has two degrees of freedom. The proximal joint includes a first end and a second end. A hollow elongated member having a longitudinal axis includes a first end and a second end, with the elongated member first end being mounted to the proximal joint second end. A distal joint constrains pivoting of parts that are mounted and adjacent to the distal joint to be about two perpendicular, intersecting axes including a primary axis and a secondary axis, such that the distal joint has two degrees of freedom. The distal joint includes a first end and a second end, with the distal joint first end being mounted to the elongated member second end. Three articulation control cable lengths include a first cable, a second cable, and a third cable that engage and operatively couple the proximal joint and distal joint to control the two degrees of freedom of the distal joint. In some such embodiments, the proximal joint is a universal joint and the distal joint is a universal joint.

In some embodiments, in any combination with the embodiments described above, the proximal joint and distal joint each include a proximal yoke at the first end, a distal yoke at the second end, a center block between the proximal yoke and distal yoke, and means for mounting the proximal yoke and the distal yoke to the center block that permit pivoting the proximal yoke and distal yoke about the two perpendicular, intersecting axes through the respective center block. In some such embodiments, the first cable and the second cable are attached to the proximal yoke of the proximal joint and to the distal yoke of the distal joint. In further embodiments, the third cable is attached to the proximal joint center block and the distal joint center block.

In some embodiments, each yoke has a first arm and a second arm that oppose each other. The first cable is attached to an arm of the proximal yoke of the proximal joint and to an arm of the distal yoke of the distal joint, the second cable is attached to an arm of the proximal yoke of the proximal joint and to an arm of the distal yoke of the distal joint, and the third cable is attached to the center block of the proximal joint and to the center block of the distal joint. Pivoting the proximal yoke of the proximal joint causes a corresponding motion of the distal yoke of the distal joint. In some embodiments, pivoting the proximal yoke of the proximal joint in a predetermined direction causes retraction of the first and second cables distally and relaxing of the third cable.

In some embodiments, the first cable and second cable follow a path partially around each center block in a first direction and the third cable follows a path partially around each center block in a second, opposite direction. In some embodiments, each center block defines a groove for receiving the third cable. In some embodiments, the center block is cylindrical with a portion having a greater radius than the remainder of the center block. In some such embodiments, the portion having a greater radius forms a proximal face and a distal face. In some such embodiments, the proximal face and the distal face are offset by approximately one quarter of the circumference of the center block. In some such embodiments, the groove is defined by the portion having a larger radius and passes through the portion having a larger radius along the circumference of the center block.

In some embodiments, in any combination with the embodiments described above, the distal yoke of the proximal joint and the proximal yoke of the distal joint each include cable guides, with each cable guide including a curved surface. In some such embodiments, the first cable and the second cable each engage a cable guide on the distal yoke of the proximal joint, the center block of the proximal joint, and a round feature on an arm of the proximal yoke of the proximal joint. The first cable and the second cable each also engage a cable guide on the proximal yoke of the distal joint, the center block of the distal joint, and a round feature on an arm of the distal yoke of the distal joint.

In some embodiments, in any combination with the embodiments described above, the three cable lengths terminate in the proximal joint and the distal joint. In some embodiments, in any combination with the embodiments described above, the surgical tool further comprises a manipulator adapted to receive at least a portion of the operator's hand, the manipulator mounted to the proximal joint first end, an end effector mounted to the distal joint second end, and one end effector actuation cable that engages and operatively couples the manipulator and the end effector. In some such embodiments, pivoting the manipulator about the primary axis of the proximal joint in a predetermined first direction at a first angle from the longitudinal axis causes the first and second cables to retract and the third cable to relax, and causes the distal yoke of the distal joint to pivot in a second direction about the primary axis of the distal joint at the same angle from the longitudinal axis and on the opposite side of the longitudinal axis as the first angle.

In some embodiments, the end effector comprises two movable jaws that operate simultaneously. In some such embodiments, the end effector further comprises a jaw actuation pin to which the end effector actuation cable is attached, wherein the jaw actuation pin is received in a slot in each jaw and proximal movement of the jaw actuation pin causes the two movable jaws to close.

In accordance with another embodiment, a surgical tool for use by an operator is provided. The surgical tool includes a proximal flexible articulation element including a first end and a second end. A tube assembly having an longitudinal axis includes a proximal tube element and a distal tube element, with the proximal tube element including a proximal end mounted to the second end of the proximal flexible articulation element and a distal end, and the distal tube element including a distal end and a proximal end rotatably mounted to the distal end of the proximal tube such that the distal tube element may rotate about the longitudinal axis relative to the proximal tube element. A distal flexible articulation element includes a first end and a second end, with the first end of the distal flexible articulation element being mounted to the distal end of the distal tube element.

In some embodiments, the surgical tool includes a manipulator adapted to receive at least a portion of the operator's hand, with the manipulator being mounted to the first end of the proximal flexible articulation element, an end effector including at least one movable jaw, with the end effector mounted to the second end of the distal flexible articulation element, and cables. The cables engage and operatively couple the manipulator, proximal flexible articulation element, and distal flexible articulation element and concurrently engage and operatively couple the manipulator and the end effector.

In some embodiments, when the distal tube element is in a first angular orientation with respect to the proximal tube element, the surgical tool is in a motion-following mode, and when the distal tube element is in a second angular orientation with respect to the proximal tube element, the surgical tool is in a motion-mirroring mode. In some embodiments, in a first mode of operation, pivoting the manipulator and the proximal flexible articulation element in a predetermined first direction at a first angle from the longitudinal axis causes the distal flexible articulation element to pivot in a second direction at the same angle from the longitudinal axis and on the opposite side of the longitudinal axis as the first angle, and in a second mode of operation, pivoting the manipulator and the proximal flexible articulation element in a predetermined first direction at a first angle from the longitudinal axis causes the distal flexible articulation element to pivot in a third direction at the same angle from the longitudinal axis and on the same side of the longitudinal axis as the first angle.

In some embodiments, in any combination with the embodiments described above, the proximal tube element includes a proximal tube including a first rotational mounting means mounted proximate to the distal end of the proximal tube element, and the distal tube element includes a distal tube including a second rotational mounting means mounted to the first rotational mounting means. In some such embodiments, the first rotational mounting means includes a flange and an annulus spaced from the flange along the longitudinal axis, and the second rotational mounting means includes a lever including a collar that is longitudinally secured and rotationally mounted to the annulus. In some such embodiments, the flange includes a distal surface that defines depressions offset by 180 degrees around the longitudinal axis, and further includes a retention means mounted to the distal tube element that engages the depressions to maintain the rotational position of the distal tube element relative to the proximal tube element. In some such embodiments, the retention means is a locking spring plunger.

In some embodiments, in any combination with the embodiments described above, the surgical tool further includes a first cable guide disposed in the proximal tube, a second guide disposed in the distal tube proximate to the end effector, and a third cable guide disposed in the distal tube between the first cable guide and the second cable guide. In some such embodiments, each of the cable guides define four holes parallel to and evenly distributed about the longitudinal axis to receive the cables. In some such embodiments, the first cable guide is in a fixed angular position relative to the proximal tube and the manipulator, and the second and third cable guides are each in a fixed angular position relative to the distal tube and each other. In some such embodiments, when the distal tube is rotated about the longitudinal axis from the first angular orientation by 180 degrees to the second angular orientation, the position of the holes and the cables extending distally from the third cable guide in the distal tube is shifted about the longitudinal axis by 180 degrees, and the end effector rotates about the longitudinal axis 180 degrees.

In some embodiments, the cables comprise four cable lengths that control both the deflection of the distal flexible articulation element and the operation of the at least one movable jaw. In some such embodiments, the four cable lengths comprise two cables terminating in the manipulator and fixed to the end effector. In some embodiments, the four cable lengths comprise four separate cables, each terminating in the manipulator and fixed to the end effector. In some such embodiments, the at least one movable jaw comprises two movable jaws that operate simultaneously.

In accordance with another embodiment, a method of operating a surgical tool is provided. The surgical tool includes a manipulator adapted to receive at least a portion of the operator's hand, a proximal flexible articulation element including a first end and a second end, with the first end of the proximal flexible articulation element being mounted to the manipulator, a tube assembly having an longitudinal axis and including a proximal tube element and a distal tube element. The proximal tube element includes a proximal end mounted to the second end of the proximal flexible articulation element and a distal end, and the distal tube element includes a distal end and a proximal end rotatably mounted to the distal end of the proximal tube element such that the distal tube element may rotate about the longitudinal axis relative to the proximal tube element. A distal flexible articulation element includes a first end and a second end, with the first end of the distal flexible articulation element being mounted to the distal end of the distal tube element. An end effector includes at least one movable jaw, with the end effector being mounted to the second end of the distal flexible articulation element. Cables engage and operatively couple the manipulator, proximal flexible articulation element, and distal flexible articulation element and concurrently engage and operatively couple the manipulator and the end effector. The method includes pivoting the manipulator and the proximal flexible articulation element in a predetermined first direction at a first angle from the longitudinal axis to cause the distal flexible articulation element to pivot in a second direction at the same angle from the longitudinal axis and on the opposite side of the longitudinal axis as the first angle. The mode of operation of the surgical tool is changed. The manipulator and the proximal flexible articulation element are pivoted in the predetermined first direction at the first angle from the longitudinal axis to cause the distal flexible articulation element to pivot in a third direction at the same angle from the longitudinal axis and on the same side of the longitudinal axis as the first angle.

In one embodiment, changing the mode of operation of the surgical tool includes rotating the distal tube element about the longitudinal axis 180 degrees relative to the proximal tube element.

Further features of a surgical tool will become more readily apparent from the following detailed description taken in conjunction with the accompanying drawings.

### BRIEF DESCRIPTION OF THE DRAWINGS

For a more complete understanding, reference should now be had to the embodiments shown in the accompanying drawings and described below. In the drawings:

FIG. 1 is a right perspective view from above of a first embodiment of a surgical tool.

FIG. 2 is a right perspective view from above of the surgical tool of FIG. 1 in a first articulated position.

FIG. 3 is a top plan view of the surgical tool of FIG. 1 in a second articulated position.

FIG. 4 is a left side view of the surgical tool of FIG. 1 in a third articulated position.

FIG. 5 is a right perspective view from above of an embodiment of a distal joint and end effector assembly of the surgical tool of FIG. 1, with the end effector in a closed position.

FIG. 6 is a right perspective view from above of an exploded view of the end effector of FIG. 5.

FIG. 7 is a right perspective view from above of an exploded view of the distal joint of FIG. 5.

FIG. 8 is a perspective view of an embodiment of the proximal yoke of the distal joint of FIG. 5.

FIG. 9 is an opposite side perspective view of the proximal yoke of FIG. 8.

FIG. 10 is an end view of the proximal yoke of FIG. 8.

FIG. 11 is a perspective view of an embodiment of the distal yoke of the distal joint of FIG. 5.

FIG. 12 is a section view of the distal yoke of FIG. 11 taken through the center and parallel to the legs.

FIG. 13 is a perspective view of an embodiment of a center block of the distal joint of FIG. 5.

FIG. 14 is an opposite side perspective view of the center block of FIG. 13.

FIG. 15 is a first section perspective view of the right side of the end effector and distal joint assembly of FIG. 5.

FIG. 16 is a second section perspective view of the top of the end effector and distal joint assembly of FIG. 5.

FIG. 17 is a third section perspective view of the left side of the end effector and distal joint assembly of FIG. 5.

FIG. 18 is a fourth section perspective view of the bottom of the end effector and distal joint assembly of FIG. 5.

FIG. 19 is a right perspective view from above of the end effector and distal joint assembly in FIG. 5 with the jaws in an open position.

FIG. 20 is a fifth section view of the end effector and distal joint assembly of FIG. 5 in the position shown in FIG. 19.

FIG. 21 is a right side exploded view an embodiment of the proximal portion of the surgical tool of FIG. 1.

FIG. 22 is a right side section view of the proximal portion of the surgical tool of FIG. 21.

FIG. 23 is a right side section view of the proximal joint of FIG. 21.

FIG. 24A is a right side section view of the proximal joint of the proximal portion of FIG. 21 in a first articulated position.

FIG. 24B is a right side section view of the articulated distal joint and end effector assembly of FIG. 5 in a position corresponding to the first articulated position of FIG. 24A.

FIG. 25A is a right side section view of the proximal joint of the proximal portion of FIG. 22 in a second articulated position.

FIGS. 25B and 25C are right side section views of the distal joint and end effector assembly of FIG. 5 corresponding to the second articulated position of FIG. 25A.

FIG. 26 is a right side view from above of a second embodiment of a surgical tool;

FIG. 27 is a right perspective view of an embodiment of a distal portion of the surgical tool of FIG. 26.

FIG. 28 is an exploded view of the distal articulation element and end effector of the distal portion of FIG. 27.

FIG. 29 is a right section view of the distal portion of the surgical tool of FIG. 27, with the end effector in an open position.

FIG. 30 is a right side view of the surgical tool of FIG. 26 in a first articulated position.

FIG. 31 is a right exploded perspective view of an embodiment of an articulation control element of surgical tool of FIG. 26.

FIG. 32 is a right section view of the articulation control element of FIG. 32.

FIG. 33A is a right section view of an embodiment of the proximal portion of the surgical tool of FIG. 30 in the first articulated position of FIG. 30.

FIG. 33B is a right section view of the distal portion of FIG. 29 of the surgical tool of FIG. 26 corresponding to the first articulated position of FIG. 33A.

FIG. 34 is a right side view of the surgical tool of FIG. 26 in a second articulated position.

FIG. 35A is a right section view of the proximal portion of FIG. 33A of the surgical tool of FIG. 26 in the second articulated position of FIG. 34.

FIG. 35B is a right section view of the distal portion of FIG. 29 corresponding to the second articulated position of FIG. 35A.

FIGS. 36A and 36B are right side views of the proximal articulation element, central portion, and distal articulation element with the central portion shortened and tube removed for illustrative purposes, in first and second positions, respectively.

FIG. 37 is a right side perspective view of an embodiment of an articulation element of the surgical tool of FIG. 26.

FIG. 38 is a top plan view of the articulation element of FIG. 37.

FIG. 39 is a right side view of the articulation element of FIG. 37.

FIG. 40 is a front view of the articulation element of FIG. 37.

### DETAILED DESCRIPTION

Embodiments of a surgical instrument are disclosed for use in a wide variety of roles including, for example, grasping, dissecting, clamping, electrocauterizing, or retracting materials or tissue during surgical procedures performed within a patient's body.

Certain terminology is used herein for convenience only and is not to be taken as a limitation. For example, words such as “upper,” “lower,” “left,” “right,” “horizontal,” “vertical,” “upward,” and “downward” merely describe relative positions or the configuration shown in the figures. The components may be oriented in any direction and the terminology, therefore, should be understood as encompassing such variations unless specified otherwise.

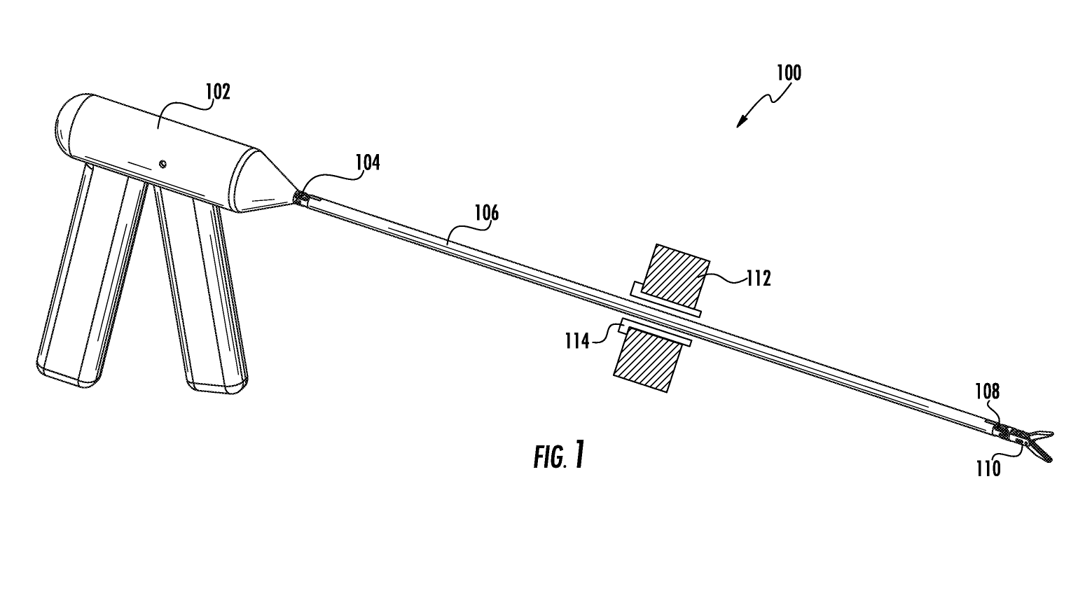

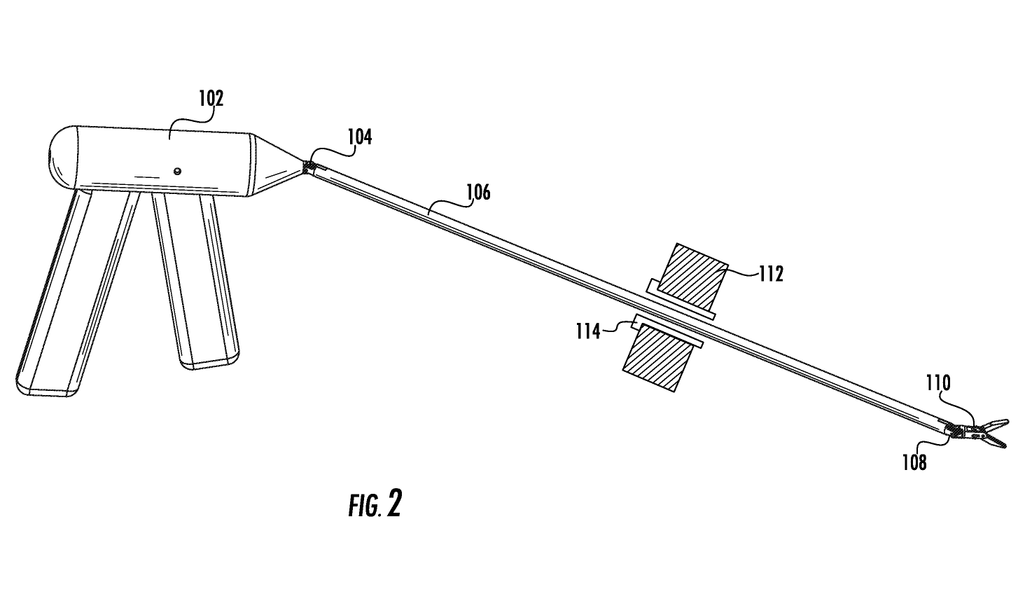

Referring now to the drawings, wherein like reference numerals designate corresponding or similar elements throughout the several views, an embodiment of a surgical tool is shown in FIGS. 1-4 and is generally designated at 100. The surgical tool 100 may include embodiments of five primary components: an operator interface or manipulator 102 designated to be at the proximal end of the tool 100, a proximal joint 104 mounted to the manipulator 102, an elongated, hollow member or tube 106 mounted to the proximal joint 104, a distal joint 108 mounted to the tube 106, and an end effector 110 mounted to the distal joint 108 and designated to be at the distal end of the tool 100. The manipulator 102 is gripped by a user's hand. The manipulator 102 and the end effector 110 may be operatively connected with cables, as discussed further below, such that when the surgeon actuates the manipulator 102, the end effector 110 has corresponding movements. The surgical tool 100 is shown in use in FIGS. 1 and 2, with a portion of the tube 106, the distal joint 108, and end effector 110 having passed through a tissue wall 112 via a cannula 114. The proximal joint 104 and the distal joint 108 may be joints that allow pivoting about two intersecting, perpendicular axes, and provide two degrees of freedom, such as, for example, a universal joint, as described in more detail below.

FIGS. 1-4 show the surgical instrument 100 in several different configurations and positions. FIG. 1 shows the instrument in its neutral position, not articulated, with the end effector 108 and manipulator 102 in an open position. The movement of the proximal joint 104 controls the movement of the distal joint 108. The proximal joint 104 and distal joint 108 are operatively connected to each other with cables, as will be discussed further below, and each of the joints 104, 108, shown as universal joints, provide two degrees of freedom, being free to move in any combination of directions deflecting from the longitudinal axis of the tube 106.

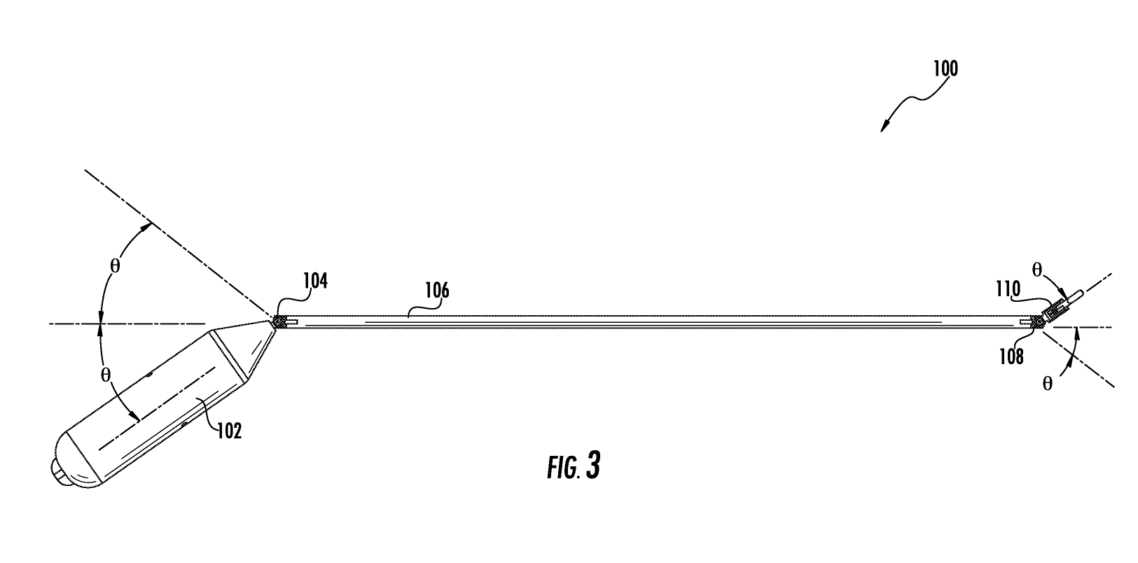

The cabling arrangement enables a surgeon to angle the manipulator 102 with his or her hand relative to the proximal joint 104 to cause the distal joint 108 to move in a similar manner in the opposite direction, imitating the surgeon's movements and providing directional control of the distal portion of the device. Such corresponding pivoted positions of the manipulator 102 and the end effector 110 relative to the longitudinal axis of the tube 106 are shown in FIGS. 3 and 4. The maximum angle of deflection θ in every direction from the longitudinal axis of the tube 106, such as side to side in FIG. 3 and between top and bottom in FIG. 4, shows the range of motion at each end of the tool 100, and is determined by the design of the proximal and distal joints 104, 108 and the direction of deflection, and may vary from the approximately 45 degrees that is shown. The tube 106 contains the cabling that operatively connects the manipulator 102 to the end effector 110 and the proximal joint 104 to the distal joint 108.

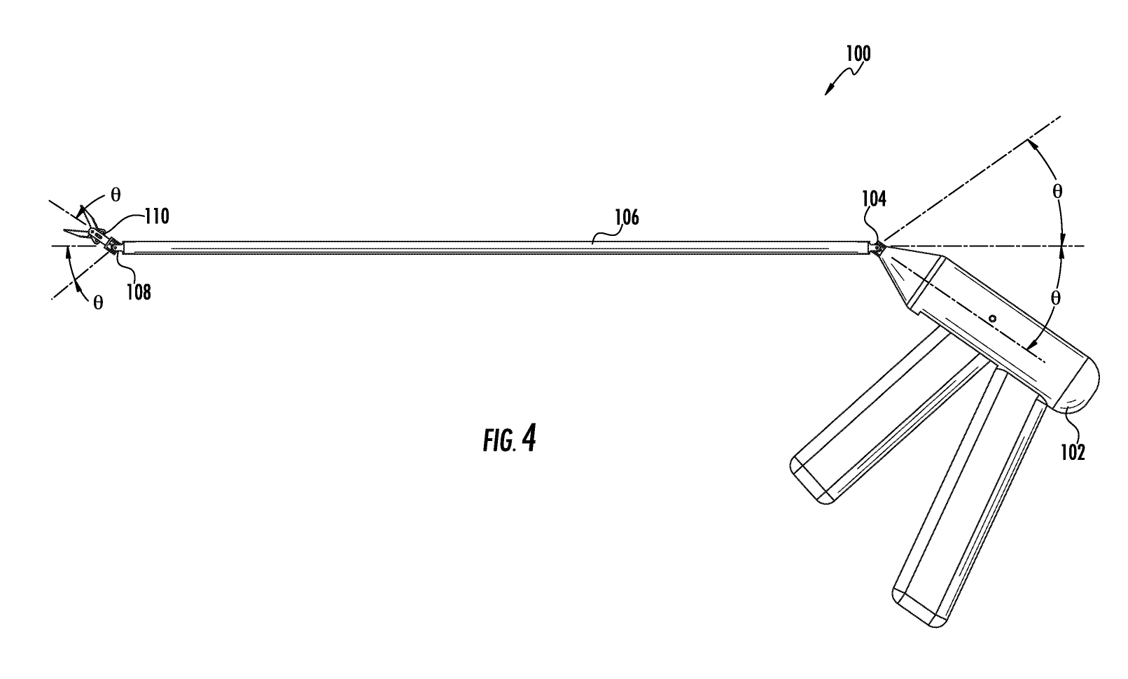

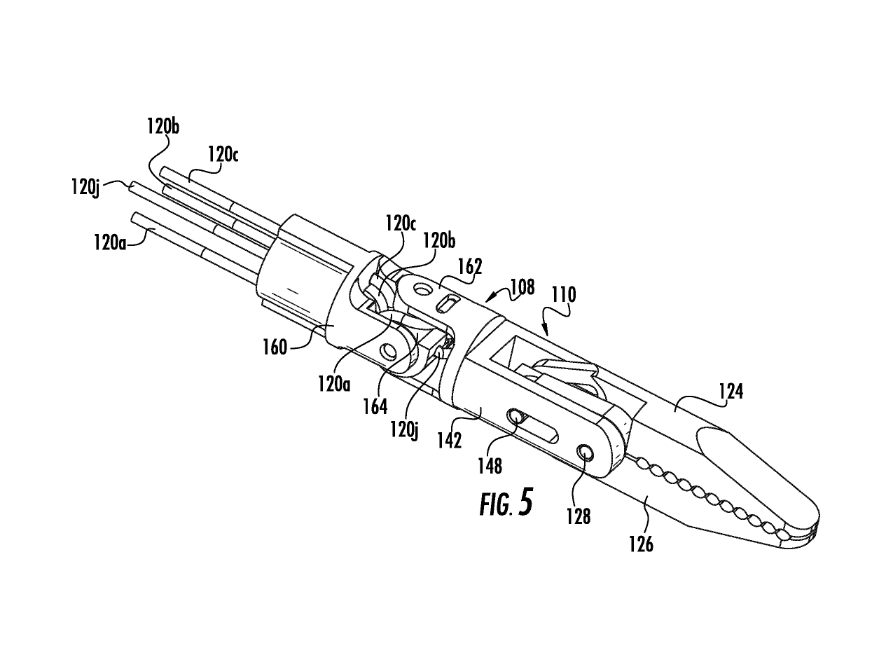

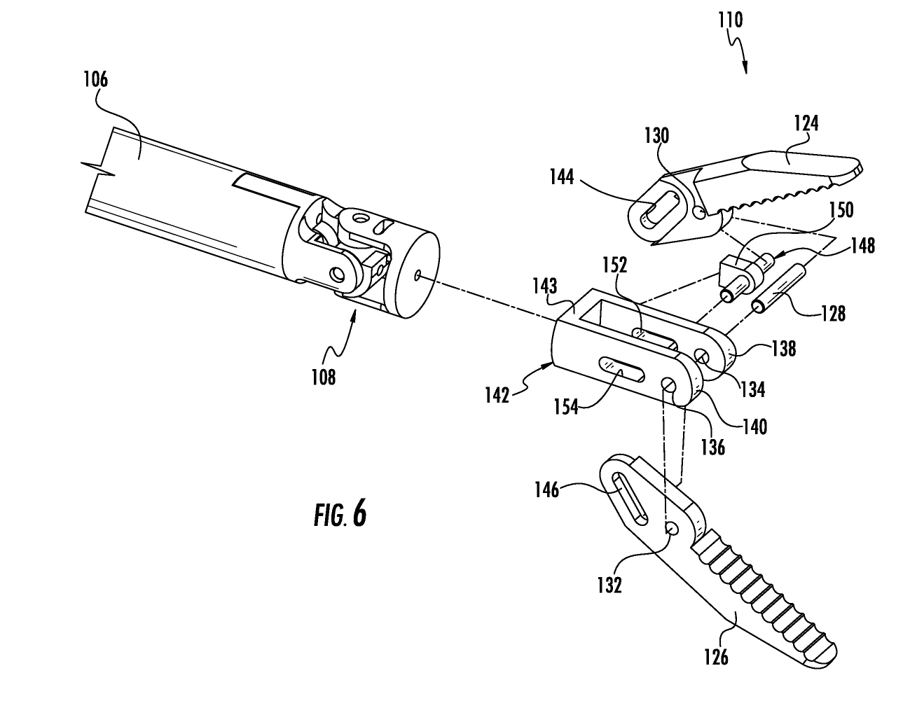

FIGS. 5-7 show the distal joint 108 and end effector 110 assembly. In FIG. 5, the tube 106 is removed to expose the cables 120a, 120b, 120c, 120j. In this embodiment, three cables 120a, 120b, 120c control the distal joint 108, and one cable 120j controls the end effector 110, which includes first and second jaw elements 124, 126. FIG. 6 shows the structure of an embodiment of the end effector 110, which may be, as shown, a jaw assembly. The first and second jaw elements 124, 126 are pivotally connected to a primary jaw pin 128 at openings 130, 132 in each jaw element 126, 128. The primary jaw pin 128 is also reciprocally received at each end in holes 134, 136 in opposing arms 138, 140 of the jaw yoke 142, which includes a base 143 between the arms 138, 140. The first and second jaw elements 124, 126 also define slots 144, 146 within which an end effector pin, referred to in this embodiment as a jaw actuation pin 148, can translate in proximal or distal directions. The jaw actuation pin 148 includes a central body portion 150 and is further received in and constrained by two slots 152, 154 in the arms 138, 140 of the jaw yoke 142. The translation of the jaw actuation pin 148 causes the jaw elements 126, 128 to rotate from a closed position to an open position and vice versa. Not shown in FIG. 6 or 7, one cable 120j that extends through the jaw yoke 142 (opening not shown in FIG. 6) controls the jaw elements 126, 128 by being connected to the central body portion 150 of the jaw actuation pin 148.

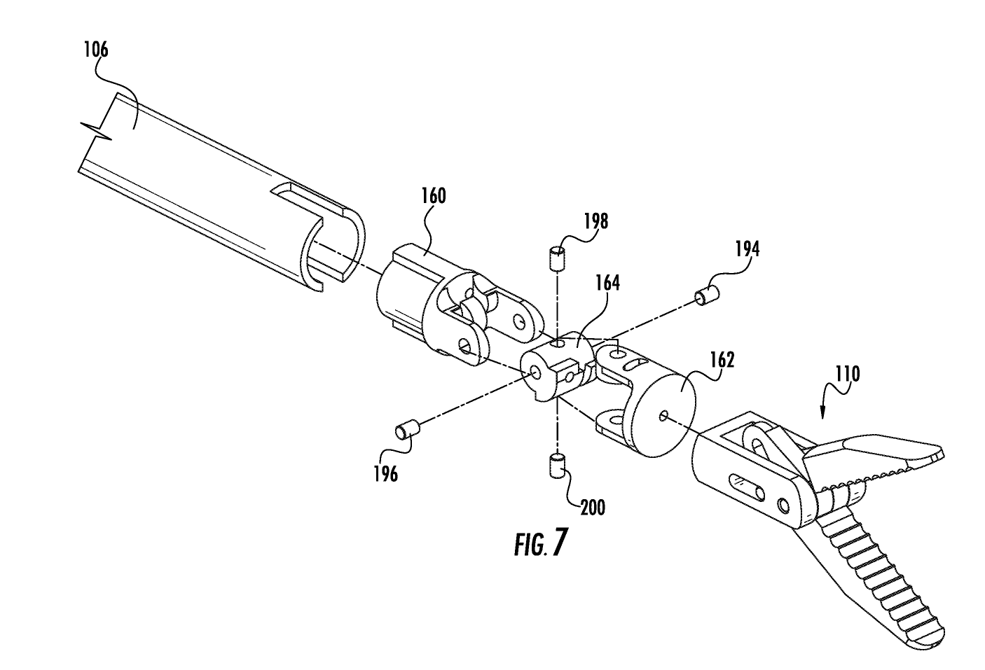

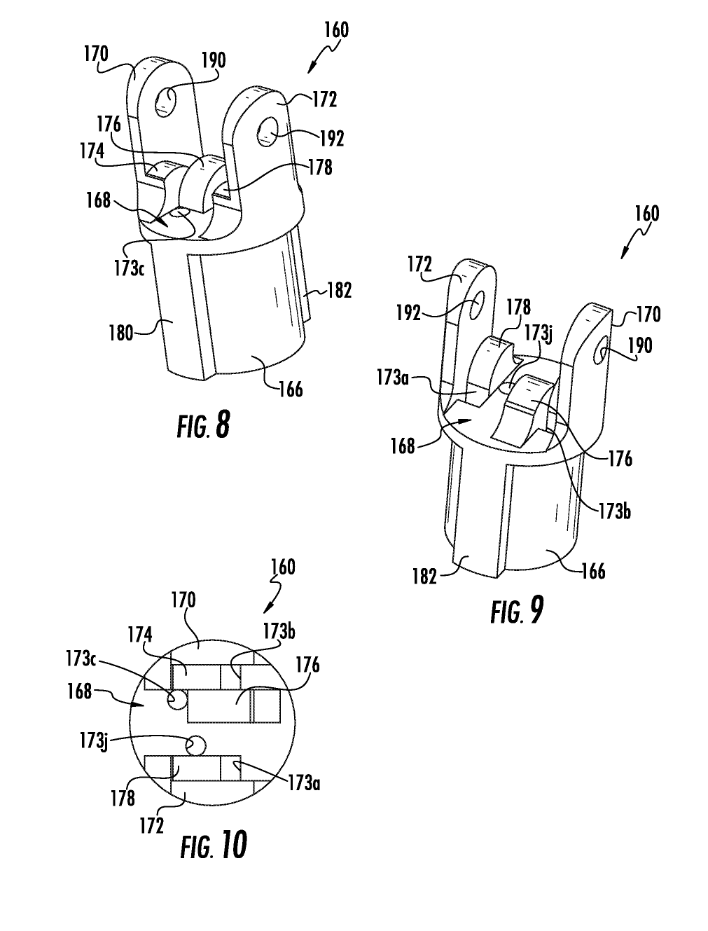

FIG. 7 shows the structure of the distal joint 108. The distal joint 108 may include a proximal yoke 160, a distal yoke 162, and a center block 164 disposed between and mounted to the proximal yoke 160 and the distal yoke 164. As shown in FIGS. 8-10, the proximal yoke 160 may include a base 166 with a distal surface 168. Two opposing arms 170, 172 extend distally from the base 166. Four holes 173a, 173b, 173c, 173j extend longitudinally through the base 166 to receive the cables 120a, 120b, 120c, 120j respectively. Three cable guides 174, 176, 178 with curved distal surfaces may project distally from the distal surface 168 of the base 166, with the cable guides 174, 176, 178 guiding the three joint control cables 120a, 120b, 120c. The proximal yoke 160 may be received in the tube section 106, and includes longitudinal protrusions 180, 182 that are received in slots 184, 186 in the tube 106 to maintain alignment. Holes 190, 192 may be provided in each arm 170, 172 to receive pins 194, 196 for pivotal mounting of the center block and that define a primary axis of articulation.

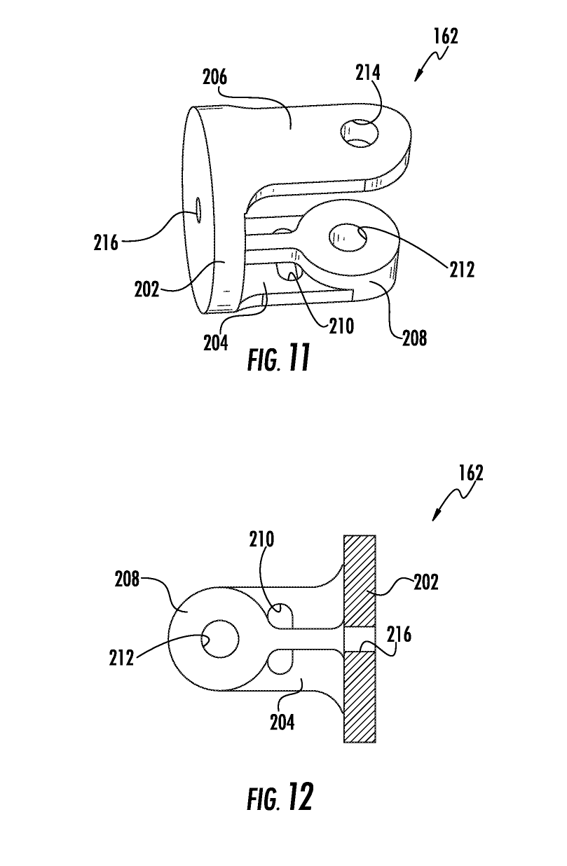

The distal yoke 162 may be pivotally connected to the center block 164 with pins 198, 200 which define a secondary axis of articulation. As shown in FIGS. 11 and 12, the distal yoke 162 may include a base 202 and two opposing arms 204, 206 that extend from the base 202. At the free end of one arm 204 a round feature 208 may be provided, as is a longitudinal slot 210 in which cables may terminate. Holes 212, 214 may be provided in each arm 204, 206 to receive the pins 198, 200. A central longitudinal opening 216 through the base 202 may be provided for passage of the end effector control cable 120j. The end effector 110 is mounted to the base 202 of the distal yoke 162.

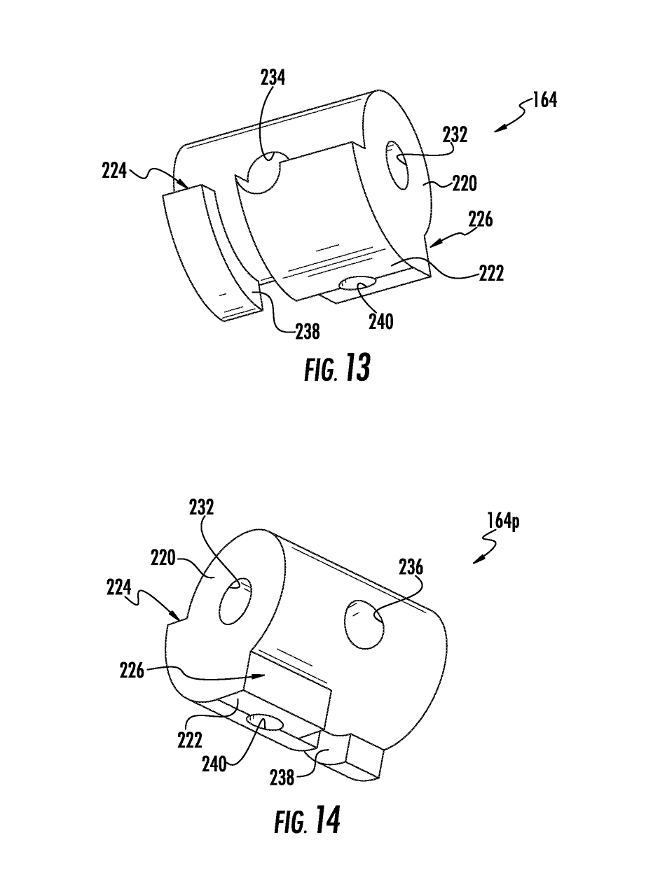

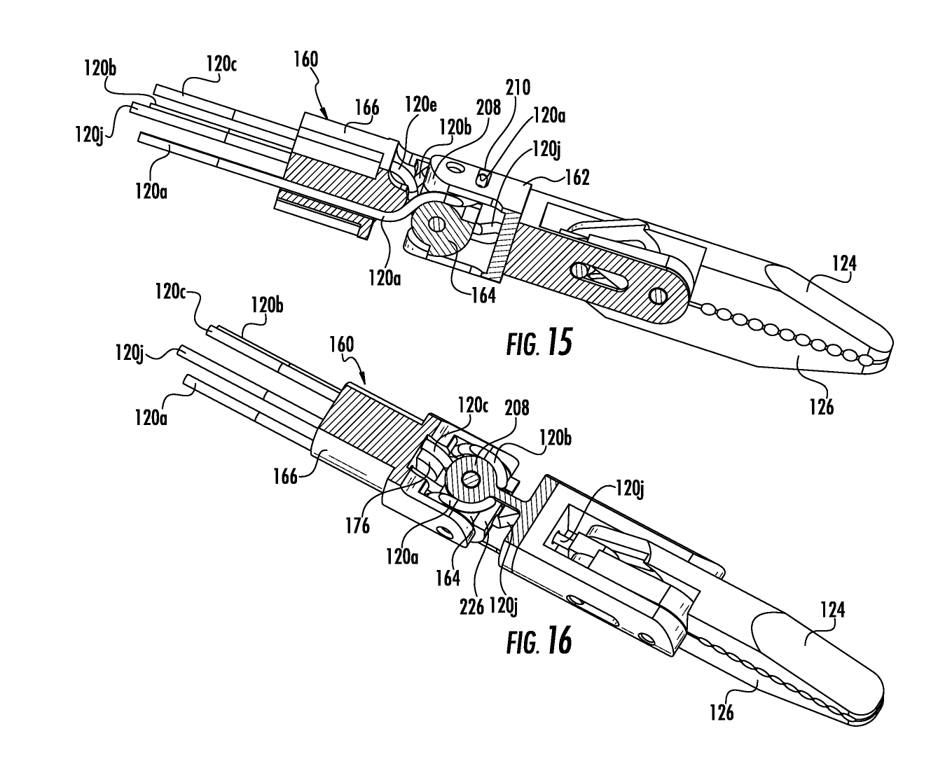

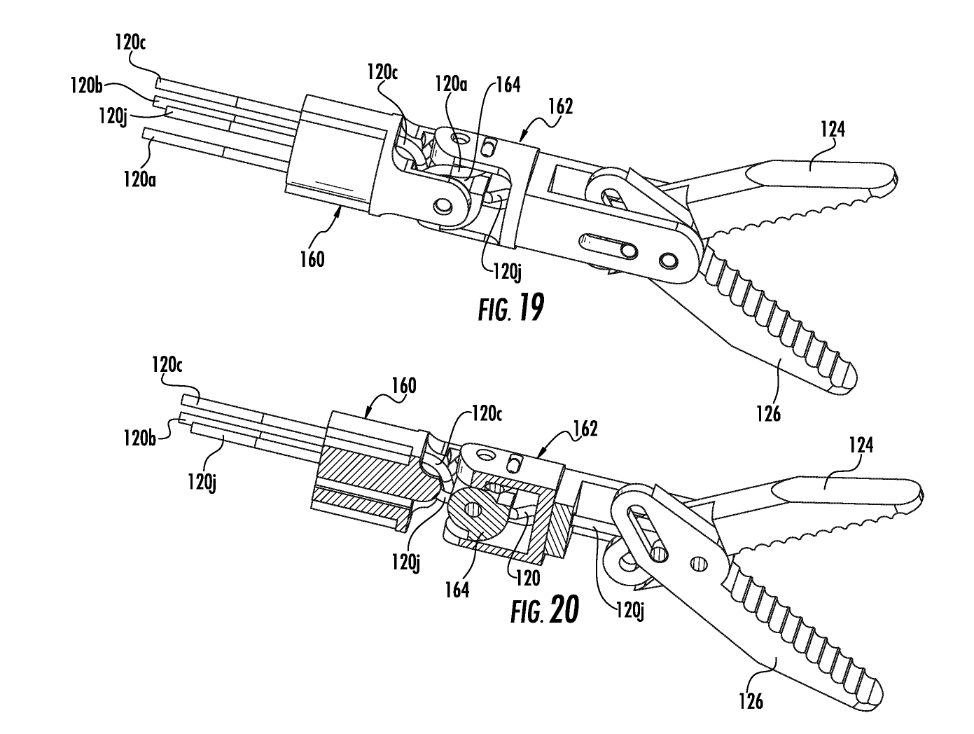

As shown in FIGS. 13 and 14, the center block 164 may include a substantially cylindrical body portion 220, and along approximately one quarter of the circumference of the body portion 220 a protrusion 222 may extend radially outward, forming a proximal face 224 and a distal face 226. Along the longitudinal axis of the center block 164, which coincides with the primary articulation axis, may be openings 230 (shown in FIG. 17), 232 that receive pins 194, 196. Perpendicular and intersecting the primary axis is the secondary axis, as positioned by openings 234, 236 in the side of the center block 164 that each receive a pin 198, 200. A groove 238 may pass circumferentially through the protrusion 222 to receive a joint control cable 120c. End effector cable 120j may pass through opening 240 through the center block 164.

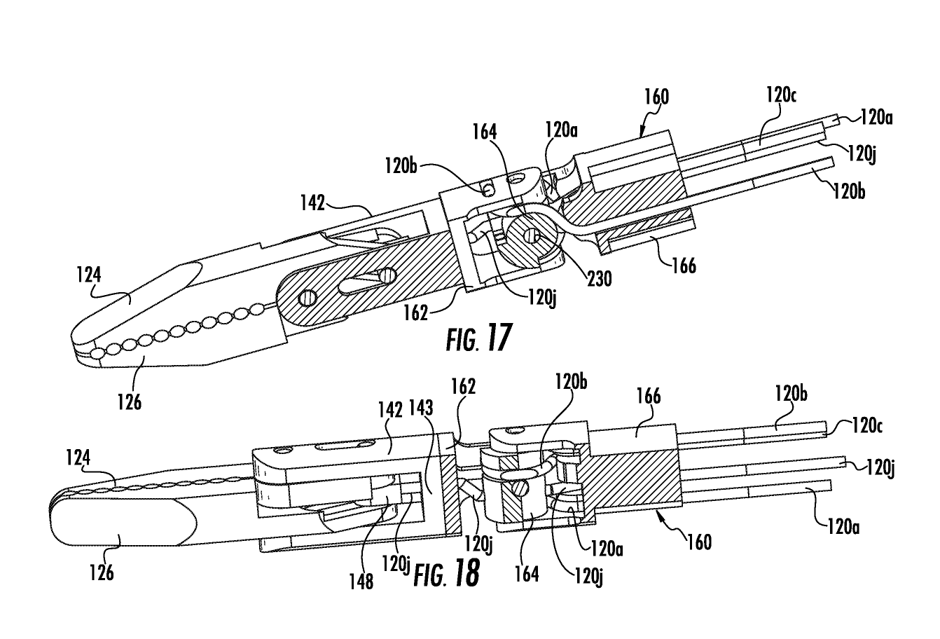

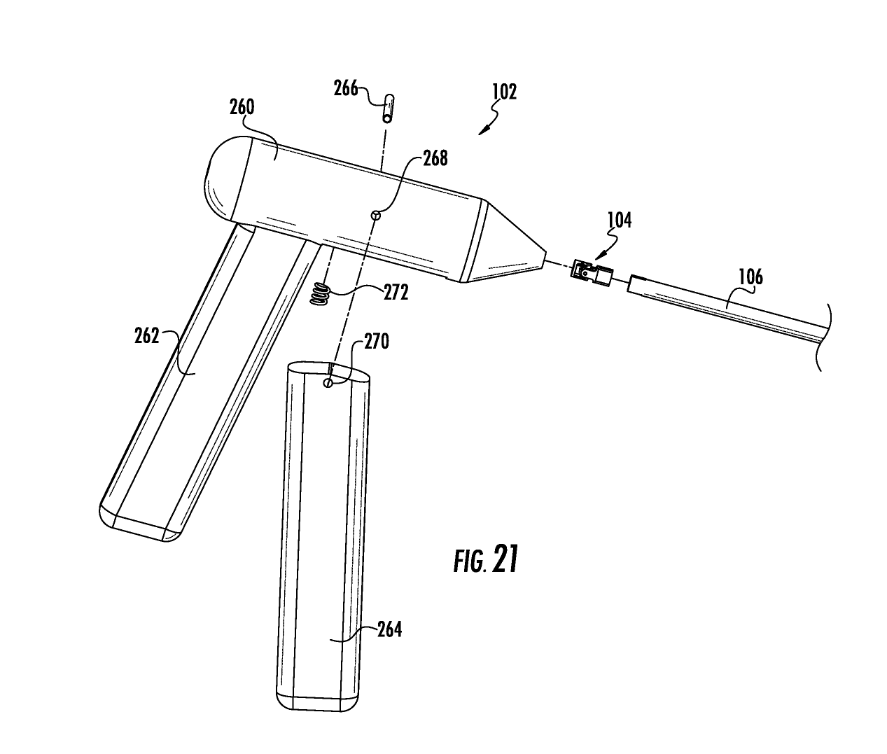

FIGS. 15-18 show the cabling that may control the distal joint 108 and end effector 110 assembly. Referring as well to the parts shown in FIGS. 8-14, cable 120a may pass under the curved surface of a cable guide 178 on the base 166 of the proximal yoke 160 and over the surface of the center block 164 where it may pass around a round feature 208 on the distal yoke 162 and terminates in the slot 210 in the distal yoke 162. Cable 120b is a mirror image of Cable 120a. Cable 120b may pass under the curved surface of a cable guide 174 on the base 166 of the proximal yoke 160 and over the surface of the center block 164 where it may pass around a round feature 208 on the distal yoke 162 and terminates in the slot 210 in the distal yoke 162. Cables 120a and 120b may connect to each other at the slot 210 as shown, forming one continuous cable. In this instance, they act as separate cables, since they are fixed to the distal yoke 162 at this point. Effectively, they terminate at the slot 210 and can be treated as separate cables for the purpose of the motion of the instrument 100. The cable 120a, 120b can be secured by means including, but not limited to, adhesive and swaging of the holes.

Cables 120a and 120b may control the articulation of the distal joint 108 and end effector 110 assembly about the secondary axis of the distal joint 108. When one of these two cables 120a, 120b is pulled while the other is relaxed, this causes an articulation about the secondary joint axis.

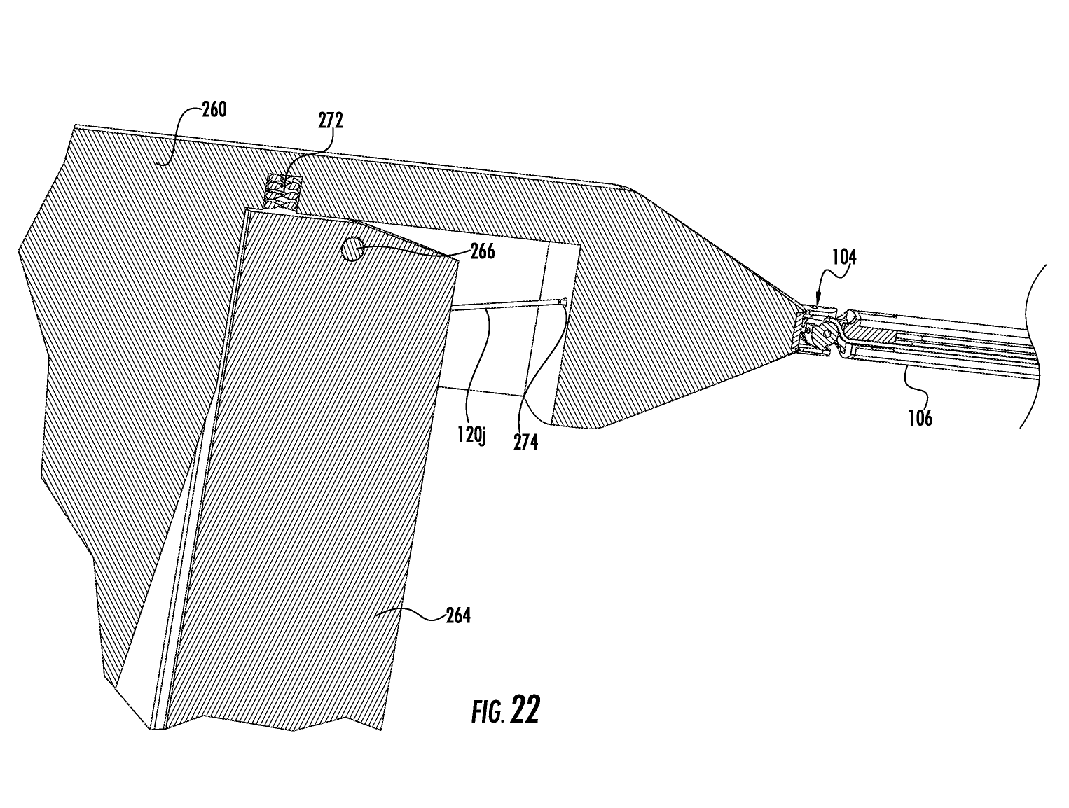

Cable 120c may pass over the curved surface of a cable guide 176 on the base 166 of the proximal yoke 160 and under the surface of the center block 164 and terminates in the groove 238 proximate to the distal face 226 of the center block 164. This cable 120c may provide tension in opposition to the other two cables 120a, 120b. If cables 120a and 120b are pulled and cable 120c is relaxed, the center block 164 and the distal yoke 162 and end effector 110 may articulate in a nominally upward direction about the primary joint axis. In this way, both axes of articulation are controlled collectively by cables 120a, 120b, and 120c. End effector control cable 120j may pass through the opening 173j in the base 166 of the proximal yoke 160, through the opening 240 in the center block 164, through the opening 216 in the base 202 of the distal yoke 162, through the opening (not shown) in the base 143 of the jaw yoke 142 to attachment at the jaw actuation pin 148. Translation of the end effector control cable 120j may cause a corresponding movement of the jaw actuation pin 148 which opens and closes the jaws 124, 126, as shown with the jaws 124, 126 open in FIGS. 19 and 20, and thus the one cable, the end effector control cable 120j, controls the one degree of freedom of the end effector 100 by being able to cause the opening and closing of the jaws 124, 126.

FIGS. 19 and 20 show the distal joint 108 and end effector 110 with jaws 124, 126 in an open position, along with the corresponding cabling.

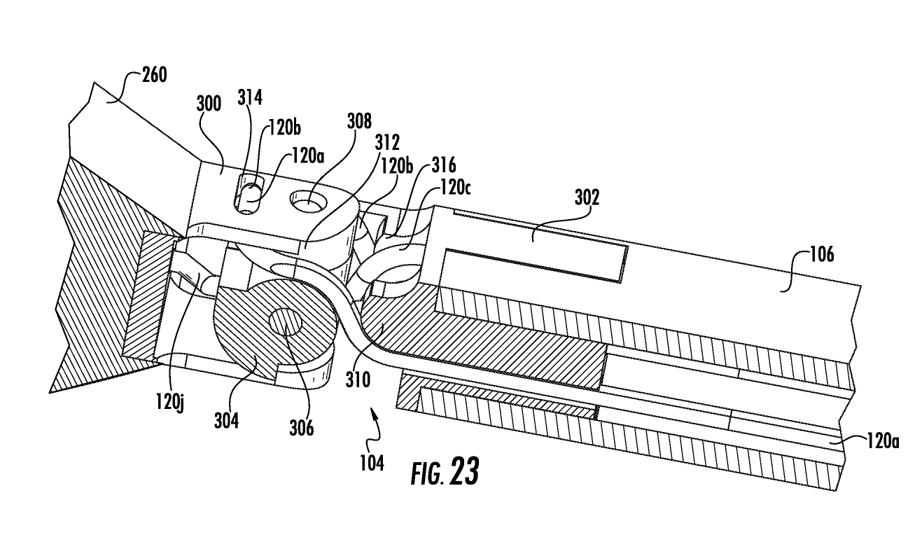

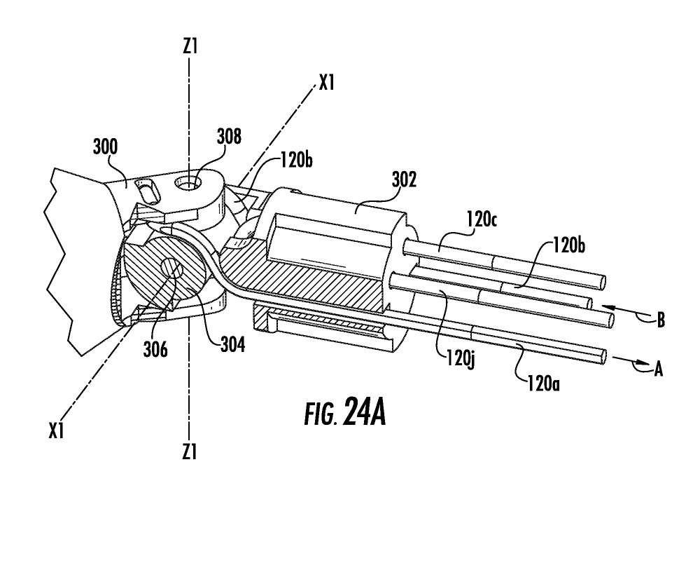

FIG. 21 shows the interface or manipulator 102 and proximal joint 104 of the surgical tool 100. The manipulator 102 may be pistol-grip style, with a body 260 and a handle 262 extending from the body 260. A trigger 264 is pivotally connected to the manipulator body 260 with a pin 266, which is received in an opening 268 in the body 260 and in an opening 270 in the trigger. A trigger spring 272 biases the trigger 264 into an open position, at which time the jaws 124, 126 of the end effector 110 are also open. As shown in FIG. 22, when the trigger 264 is pulled toward the handle 262, the spring 272 is compressed, and the trigger 264 pivots about the pin 266. The end effector control cable 120j passes through an opening 274 in the body 260 and is attached to the trigger 264, such the when the trigger 264 is pulled the cable 120j is pulled to urge the jaws 124, 126 closed. The distal end of the body 260 is connected to the proximal joint 104, which is mounted to the tube section 106.

FIG. 23 shows the proximal joint 104. The proximal joint 104 may be substantially a mirror image of the distal joint 108 about a plane perpendicular to the longitudinal axis of the tube 106. The proximal yoke 300 of the proximal joint may be similar to and substantially a mirror image of the distal yoke 162 of the distal joint 108, and the distal yoke 302 of the proximal joint 104 may be similar to substantially a mirror image of the proximal yoke 160 of the distal joint 108. Likewise, the center block 304 of the proximal joint 104 may be similar to and substantially a mirror image of the center block 164 of the distal joint 108. The proximal yoke 300 of the proximal joint 104 may pivot about two perpendicular, intersecting axes through the center block 304. The primary axis may be established by a pin 306 and a corresponding pin on the opposite side of the center block (not visible in FIG. 23), and a secondary, perpendicular axis may be established by a pin 308 and a corresponding pin (also not shown in FIG. 23) on the opposite side of the center block 304.

Referring to the cabling arrangement in FIG. 23, cable 120a may pass under a guiding surface 310 on the distal yoke 302 and over the surface of the center block 304 where it may pass around a round feature 312 on the proximal yoke 300 and terminates in the proximal yoke 300 at a slot 314. Cable 120b may be a mirror image of cable 120a. Cable 120b may pass under a guiding surface 316 on the distal yoke 302 and over the surface of the center block 304 where it passes around the round feature 308 on the proximal yoke 300 and terminates in the proximal yoke 300 at the slot 314. Cables 120a and 120b may be driven by the articulation of the proximal yoke 300 and manipulator 102 about the secondary axis of the proximal joint 104. Cable 120c may pass under the center block 304 and terminates in the groove proximate to the proximal face of the center block 304. This cable 120c may provide tension in opposition to cables 120a and 120b.

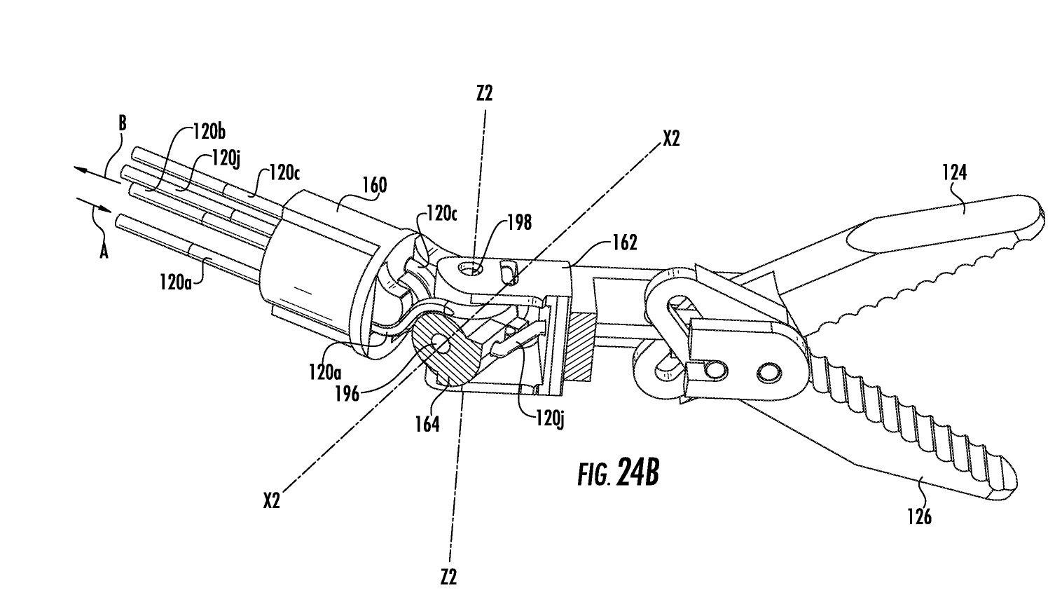

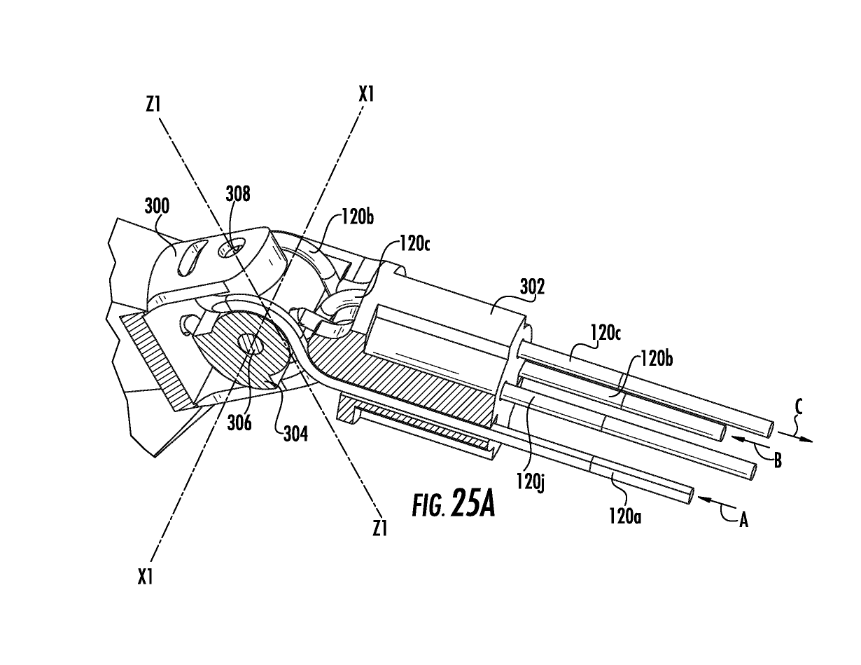

FIGS. 24A and 24B show corresponding articulated positions of the proximal joint 104 and the distal joint 108 with articulation about the secondary axis Z1-Z1 of each joint 104, 108. In this example there is no articulation about the primary axis X1-X1. In FIG. 24A, the manipulator 102 is pivoted to the right, and the proximal yoke 300 of the proximal joint 104 follows to the right. This results in tensioning of cable 120b, with a force B exerted on cable 120b, and relaxing of cable 120a, with movement of cable 120a in the direction of arrow A. Force B pulls cable 120b, pulls the distal yoke 162 of the distal joint 108, and causes that distal yoke 162 to pivot to the left about the secondary axis Z2-Z2. The end effector 110 follows to the left. In this example, cable 120c is unaffected, and there is no articulation at the distal yoke 162 of the distal joint 108 about the primary axis X2-X2. Pivoting the manipulator 102 to the left has the opposite effect.

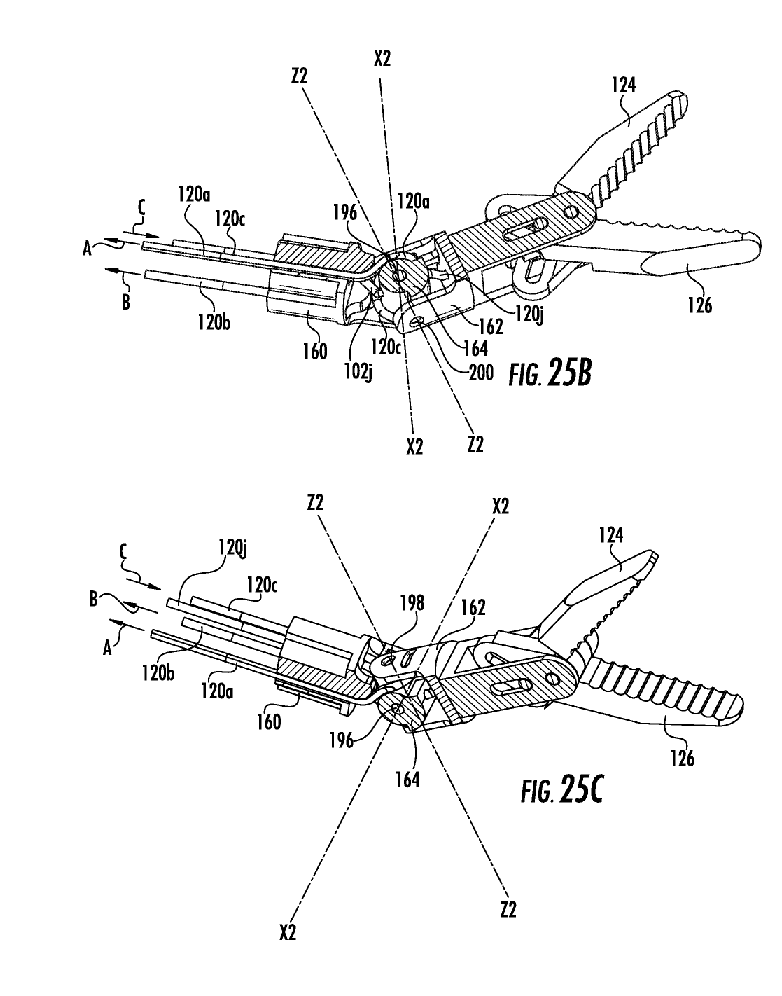

FIGS. 25A, 25B, and 25C show corresponding articulated positions of the proximal joint 104 and the distal joint 108 with articulation about the primary axis X1-Z1 of each joint 104, 108. In this example there is no articulation about the secondary axis Z1-Z1. In FIG. 25A, the manipulator 102 is pivoted downward, and the proximal yoke 300 of the proximal joint 104 follows downward. This results in tensioning of cables 120a and 120b, with a force A exerted on cable 120a, and with a force B exerted on cable 120b. Cable 120c is relaxed, with movement of cable 120c in the direction of arrow C. FIGS. 25B and 25C are offset views of the same position of the distal yoke 164 of the distal joint 108. Force A pulls cable 120a, force B pulls cable 120b, both force A and B pull the distal yoke 162 of the distal joint 108, and causes that distal yoke 162 to pivot upward about the primary axis X2-X2. The end effector 110 follows upward. In this example there is no articulation at the distal yoke 162 of the distal joint 108 about the secondary axis Z2-Z2. Pivoting the manipulator 102 upward has the opposite effect.

Both axes of articulation are controlled collectively by cables 120a, 120b, and 120c. Accordingly, the three distal joint control cables 120a, 120b, 120c provide control of the two degrees of freedom of the distal joint 108. The end effector 110 design is meant to be applicable to any assembly utilizing a single cable for actuation and producing motion of a plurality of objects, which are referred to herein as jaws, and include but are not limited to cauterizing contacts, pliers, and scissor blades.

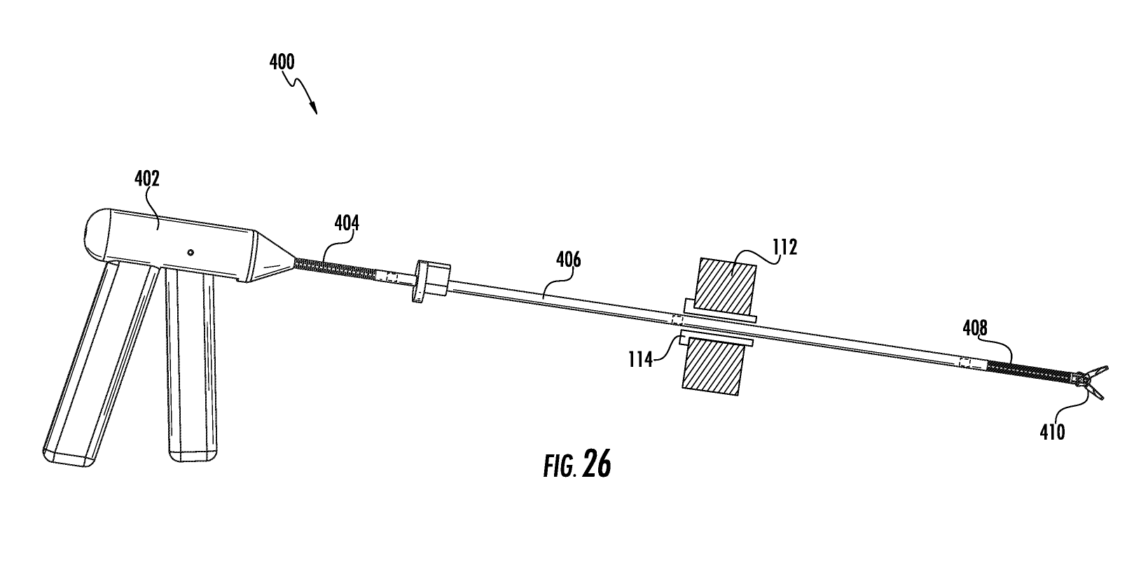

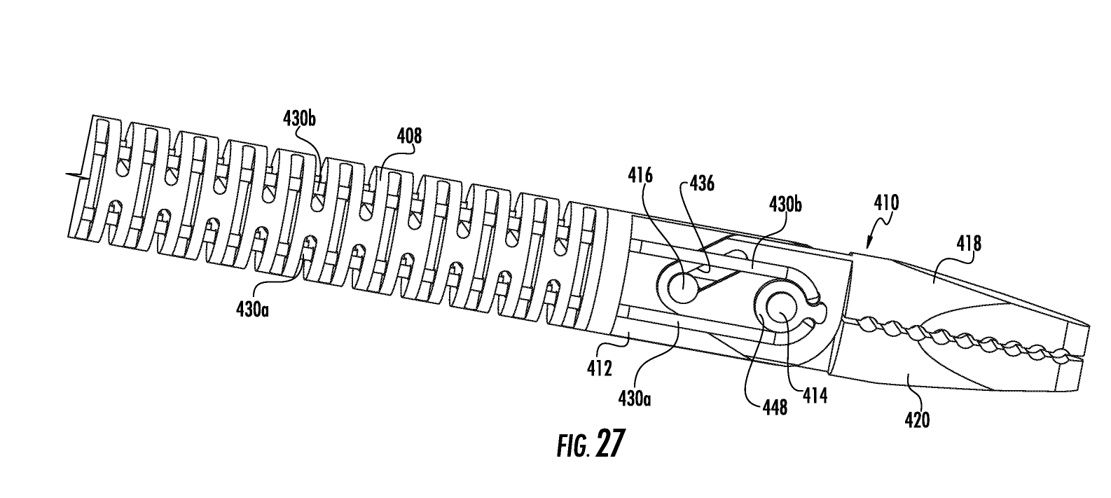

Another embodiment of a surgical tool is shown in FIG. 26 and is generally designated at 400. The surgical tool 400 may include embodiments of five primary components: an operator interface or manipulator 402 designated to be at the proximal end of the tool 400, a proximal flexible articulation element 404 mounted to the manipulator 402, an elongated, hollow member assembly or tube assembly 406 mounted to the proximal flexible articulation element 404, a distal flexible articulation element 408 mounted to the tube assembly 406, and an end effector 410 mounted to the distal flexible articulation element 408 and designated to be at the distal end of the tool 400. The manipulator 402 may be gripped by a user's hand. The manipulator 402 and the end effector 410 may be operatively connected with cables, as discussed further below, such that when the surgeon actuates the manipulator 402, the end effector 410 may have corresponding movements. The surgical tool 400 is shown in use in FIG. 26, with a portion of the tube assembly 406, the distal articulation element 408, and the end effector 410 having passed through a tissue wall 112 via a cannula 114.

FIG. 26 shows the instrument in its neutral position, not articulated, with the end effector 408 and manipulator 402 in an open position. The movement of the proximal articulation element 404 controls the movement of the distal articulation element 408. The proximal and distal articulation elements 404, 408 may be operatively connected to each other with cables, as will be discussed further below, and are free to move in directions deflecting in a substantially smooth curve away from the longitudinal axis of the tube assembly 406.

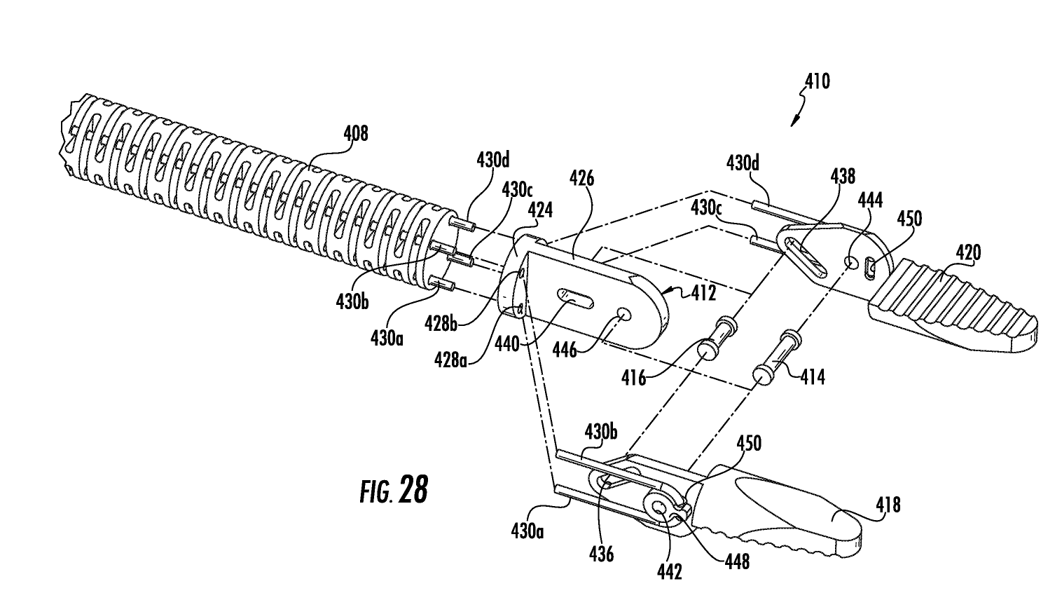

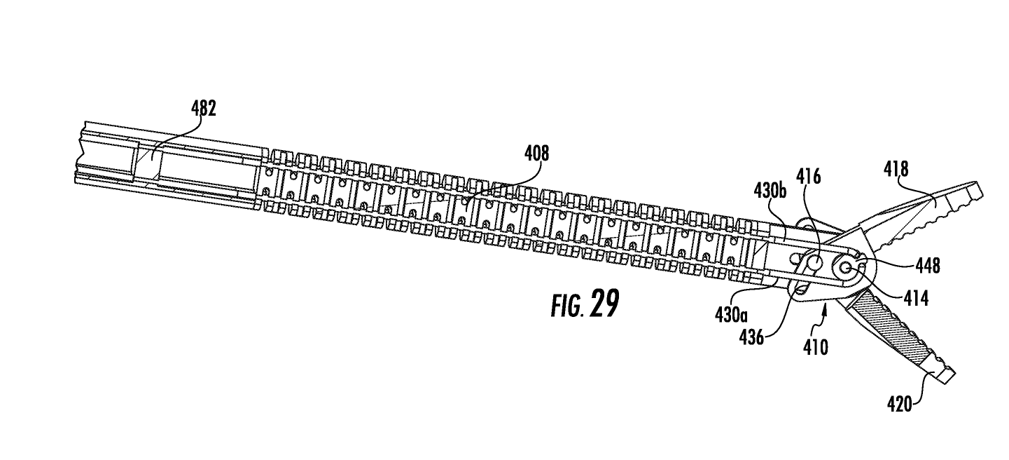

FIGS. 27-29 illustrate the structure and control of the end effector 410, which in the embodiment shown may be a jaw assembly, with the end effector 410 in a closed position. The end effector 410 may include a base 412, a rotation pin 414, constraining pin 416, and two jaws 418, 420. In this embodiment, each of the jaws 418, 420 has the same design as the other. The base 412 may include a flange 424 at the proximal end and a central portion 426 extending distally. Four holes 428a, 428b (two holes are not shown in FIGS. 27-29, but are placed similarly to holes 428a, 428b on the opposite side of the central portion 426) through the flange 424 each receive a cable 430a, 430b, 430c, 430d. The constraining pin 416 is disposed in slots 436, 438 in the respective jaws 418, 420 as well as the slot 440 in the central portion 426 of the base 412, which is between the jaws 418, 420. The rotation pin 414 is distal from the constraining pin, and is disposed in openings 442, 444 in the respective jaws 418, 420 as well as the opening 446 in the central portion 426 of the base 412. Round features 448 are provided on each jaw 418, 420 around the openings 442, 444. There is a slot 450 at the distal side of each of the round features 448.

The jaws 418, 420 may be prevented from rotating in the same direction by the constraining pin 416. When the top jaw 418 is rotated counterclockwise, the constraining pin 416 is actuated in a distal direction. When the bottom jaw 420 is rotated counterclockwise, the constraining pin 416 is actuated in a proximal direction. These actions can not occur concurrently, and thus the jaws 418, 420 are constrained to move opposite one another. As will be seen below, this actuation system is what allows the jaws 418, 420 to be controlled without interfering with articulation control.

The two jaws 418, 420 may be controlled by the four cables 430a, 430b, 430c, 430d. Cables 430a and 43b are connected to the bottom and top respectively of the round feature 448 on the bottom jaw 418. Cables 430c and 430d are connected similarly to the other jaw 420. The cables 430a, 430b, 430c, 430d may terminate and be attached in the slots 450 near the round features 448 in each jaw 418, 420. When cable 430a is retracted and cable 430b is relaxed, this actuates the top jaw 420 to a closed position. When cable 430d is retracted and cable 430c is relaxed, the bottom jaw 418 is actuated to a closed position.

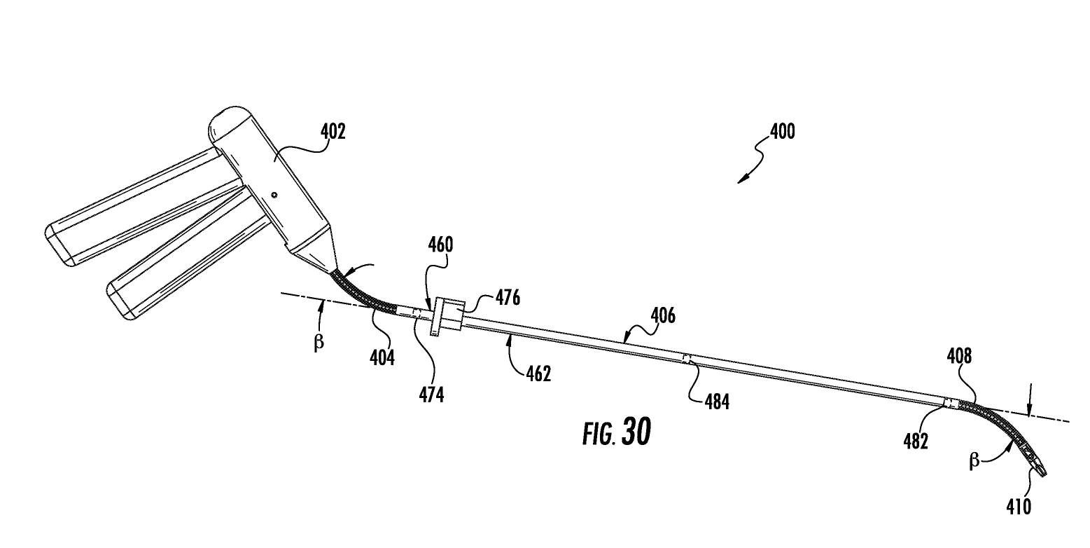

FIG. 30 shows the instrument 400 articulated by pivoting the manipulator 402 upward. In this embodiment, the instrument 400 has two modes for controlling the motion of the distal end. The first mode, which is shown in FIG. 30, is a motion-following mode, in which the distal end of the instrument 400 follows the motion of the proximal end. When the manipulator 402, at the proximal end, is deflected away from the longitudinal axis by an angle β, the distal end deflects at substantially the same angle β in the opposite direction away from the longitudinal axis of the tube assembly 406. In the other mode of operation, which is a motion-mirroring mode and is discussed further below, the distal end of the instrument 400 mirrors the motion of the proximal end and deflects in the same direction away from the longitudinal axis of the tube assembly 406 as the manipulator 402. Although the articulation is shown to be in the vertical plane, it should be understood that the instrument 400 may articulate in any direction away from the longitudinal axis within the range of movement allowed by the articulation elements 404, 408.

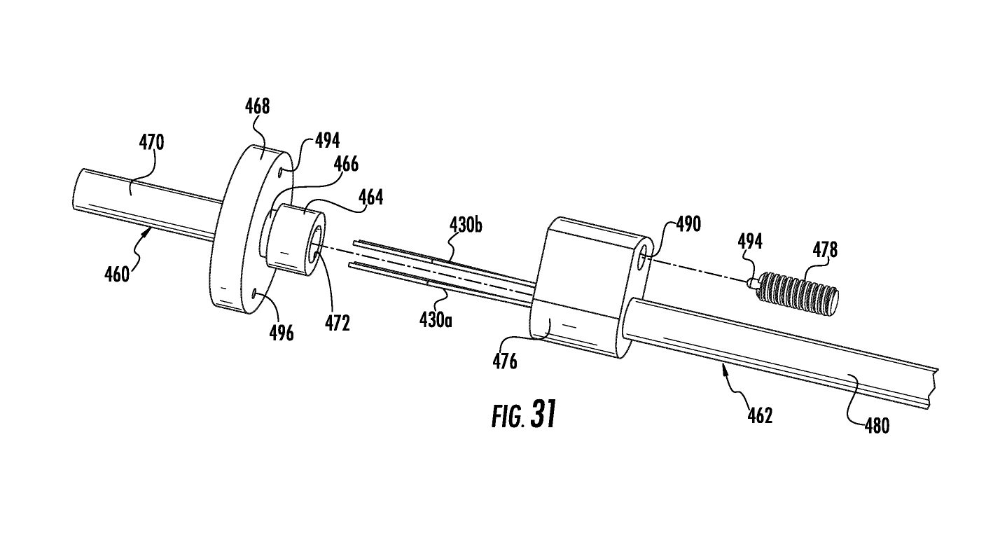

As shown in FIGS. 31 and 32, the tube assembly 406 may include a proximal tube element 460 mounted to a distal tube element 462, which forms an articulation control element. The proximal tube element 460 may include a locking annulus 464 at the distal end, with a neck 466 having a reduced diameter adjacent to the locking annulus 464, a flange 468 adjacent to the neck 466, and a proximal tube 470 adjacent to the flange 468. An opening 472 extends from the distal end to the proximal tube 470, and a cable guide 474 may be disposed in the proximal tube 470. The distal tube element 462 may include a lever 476 at the proximal end, a locking spring plunger 478, and a distal tube 480. Two cable guides 482, 484 may be disposed in the distal tube 480, with one cable guide 482 proximate to the distal articulation element 408 and the other cable guide 484 approximately centered between the other cable guides 474, 482. The lever 476 includes an opening 486 along the same longitudinal axis as the opening 472 in the proximal tube element 460 for passage of the cables 430a, 430b, 430c, 430d. The opening 486 becomes larger to receive the locking annulus 464 of the proximal tube element 460, with a collar 488 to secure the locking annulus 464 in place and to be disposed in the groove adjacent to the neck 466 of the proximal tube element 460. The lever 476 including the collar 488 and may be made in separate components, for example, which are pressed on to the annulus 464, or the assembly may be cast as one item with removable filler material to keep the parts distinct. A threaded opening 490 in the lever 476 receives the locking spring plunger 478.

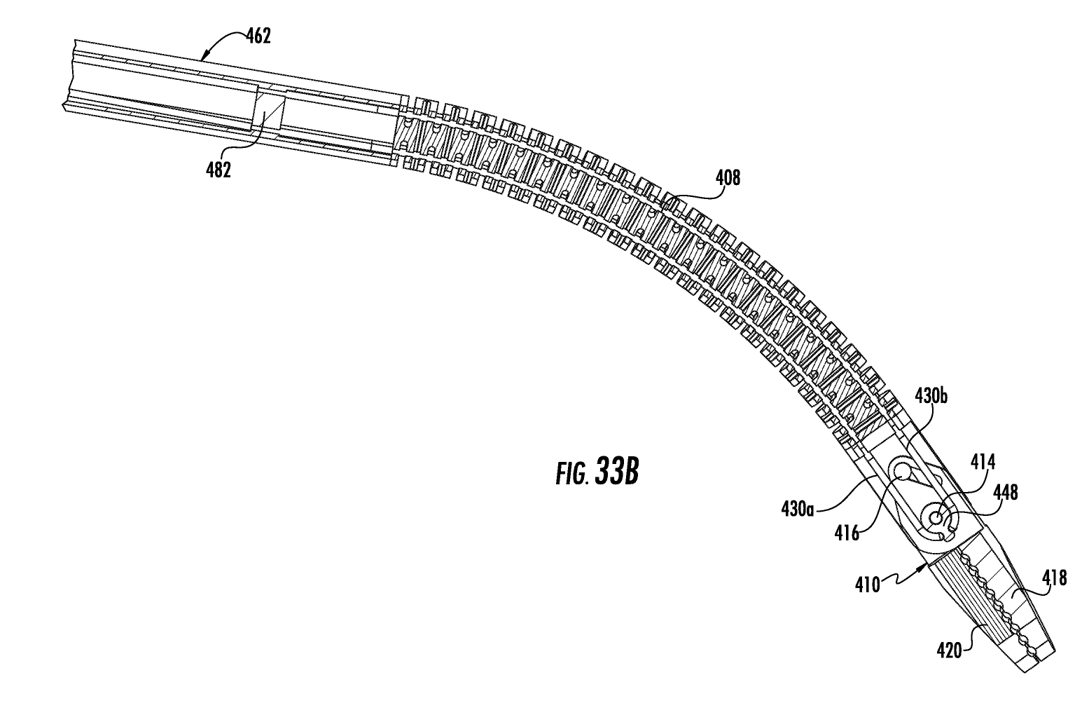

In the embodiment shown, with the lever 476 in the “up” position as in FIGS. 30-32 and 33A, the instrument 400 is in the motion-following mode. The lever 476 may rotate around the distal tube 480 and the locking annulus 464. When the lever 476 is rotated to the “down” position (as shown in FIG. 34), the instrument 400 is in the motion-mirroring mode. The flange 468 of the proximal tube element 460 has two depressions 494, 496 that are 180 degrees apart on its distal face. The depressions 494, 496 receive the biased tip 498 of the locking spring plunger 478 to releasably secure the lever 476 in position. Accordingly, a user may switch from the motion-following to the motion-mirroring control mode by rotating the lever 476 clockwise (facing distally; from the user's perspective), which rotates the entire distal tube element 462, until the locking spring plunger 478 is received in the bottom depression 496 of the flange 468 to maintain the rotational orientation of the distal tube element. Likewise, a user may switch from the motion-mirroring to the motion-following control mode by rotating the lever 476 counterclockwise (again facing distally and from the user's perspective), which rotates the entire distal tube element 462, until the locking spring plunger 478 is received in the top depression 494 of the flange 468. The change in modes is the result of altered orientations of cables 430a, 430b, 430c, 430d caused by the rotation of the distal tube element 462 and its cable guides 482, 484 relative to the proximal tube element 460 and its cable guide 474, as will be discussed further below.

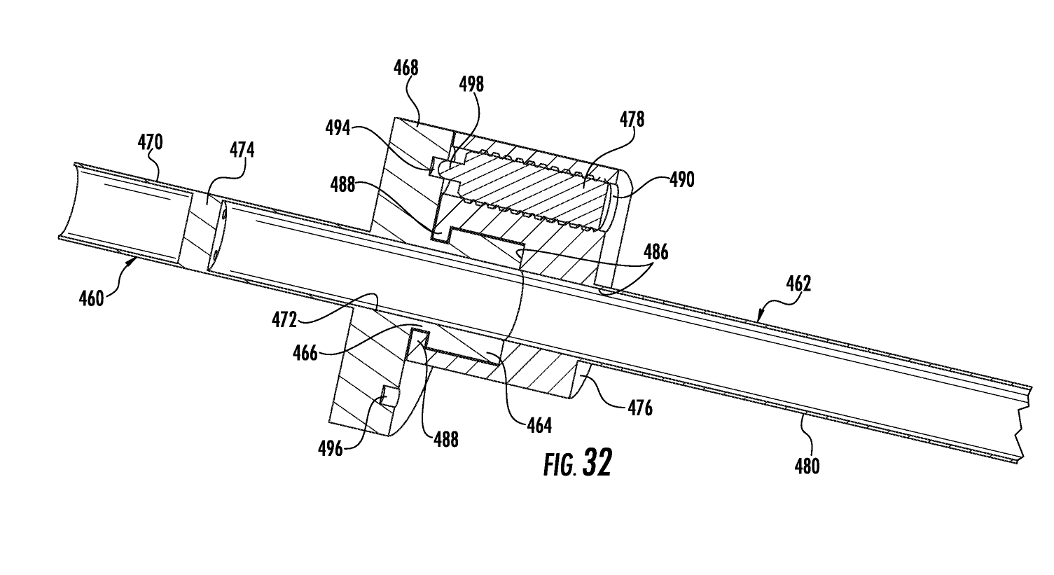

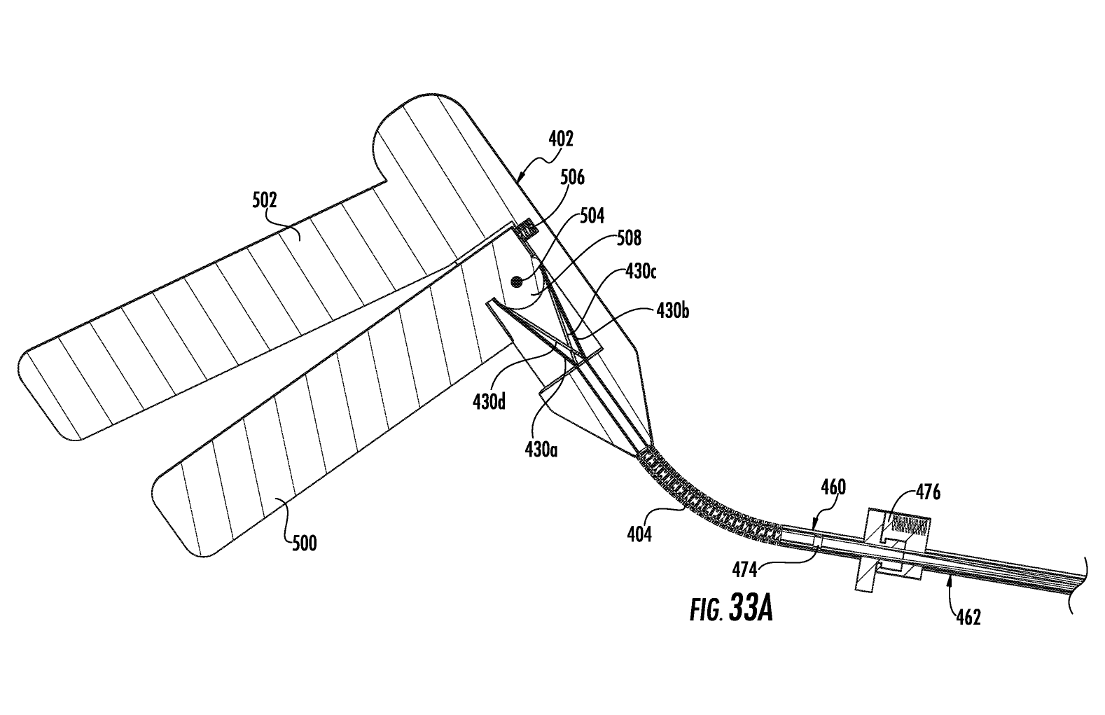

FIGS. 33A and 33B show the proximal and distal ends of the instrument 400 in corresponding positions while in the motion-following control mode. Lever 476 is in the “up” position. When cables 430a and 430b are retracted by an upward movement of the manipulator 402 (FIG. 33A), this would rotate both jaws 418, 420 in a clockwise direction, but that rotation is prevented by the constraining pin 416. As a result, the jaws 418, 420 lock against the constraining pin 416 and the distal articulation element 408 is bent downward (FIG. 33B). Due to the configuration of the cables as they terminate in the trigger 500 of the manipulator 402, the movement of the manipulator 402 has no impact on the trigger 500. Similarly, retraction of cables 430b and 430d by a downward movement of the manipulator 402 produces an upward movement of the distal articulation element 408, and retraction of either cables 430a and 430b or 430c and 430d produces lateral movement. Each of these motions is produced by a corresponding control motion of the manipulator 402 that directs the orientation of the end effector 410 with respect to the manipulator 402.

FIG. 33A shows the connections between the control cables 430a, 430b, 430c, 430d and the manipulator 402. The manipulator 402 may include a base 502, trigger 500, trigger pin 504, and spring 506. The spring 506 biases the trigger 500 into an open position as the trigger 500 pivots about the trigger pin 504. Cables 430a and 430d may terminate at the bottom of a round feature 508 on the trigger 500 while cables 430b and 430c may terminate at the top of this round feature. When the trigger 500 is actuated by the user into a closed position, cables 430a and 430d are retracted and cables 430b and 430c are relaxed, actuating the jaws 418, 420 to a closed position.

FIG. 34 shows the result of a mode change to motion-mirroring control mode, in which an action by the manipulator 402 is mirrored in the position of the end effector 410. In this case, an upward movement of the manipulator 402 causes an upward movement of the end effector 410. The end effector 410 is in a position that is a mirror image of where it would be if its orientation relative to the manipulator 402 was preserved, as in the motion-following control mode. When the manipulator 402, is deflected away from the longitudinal axis of the tube assembly 406 by an angle β, the distal end deflects at substantially the same angle β in the same direction away from the longitudinal axis.

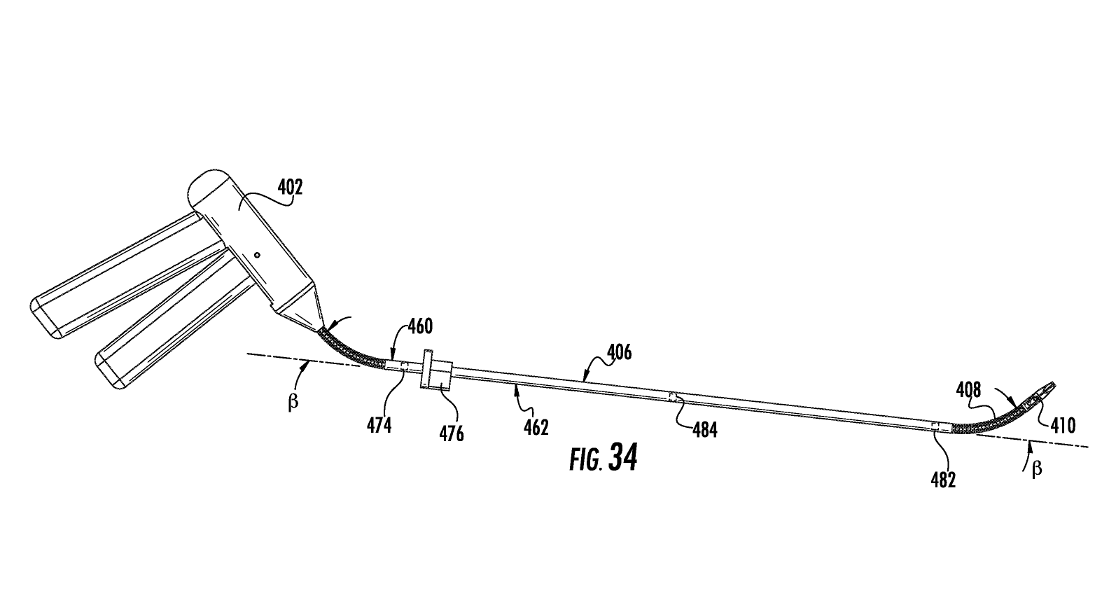

FIGS. 35A and 35B show the proximal and distal ends of the instrument 400 in corresponding positions while in the motion-mirroring control mode. Lever 476 is in the “down” position. As in the motion-following mode, when cables 430a and 430b are retracted by an upward movement of the manipulator 402 (FIG. 35A), this would rotate both jaws 418, 420 in a clockwise direction, but that rotation is prevented by the constraining pin 416. As a result, the jaws 418, 420 lock against the constraining pin 416 and the distal articulation element 408 is bent upward (FIG. 35B). Similarly, retraction of cables 430b and 430d by a downward movement of the manipulator 402 produces a downward movement of the distal articulation element 408, and retraction of either cables 430a and 430b or 430c and 430d continues to produce lateral movement in the same direction away from the longitudinal axis of the tube assembly 406 as the manipulator 402 is moved.

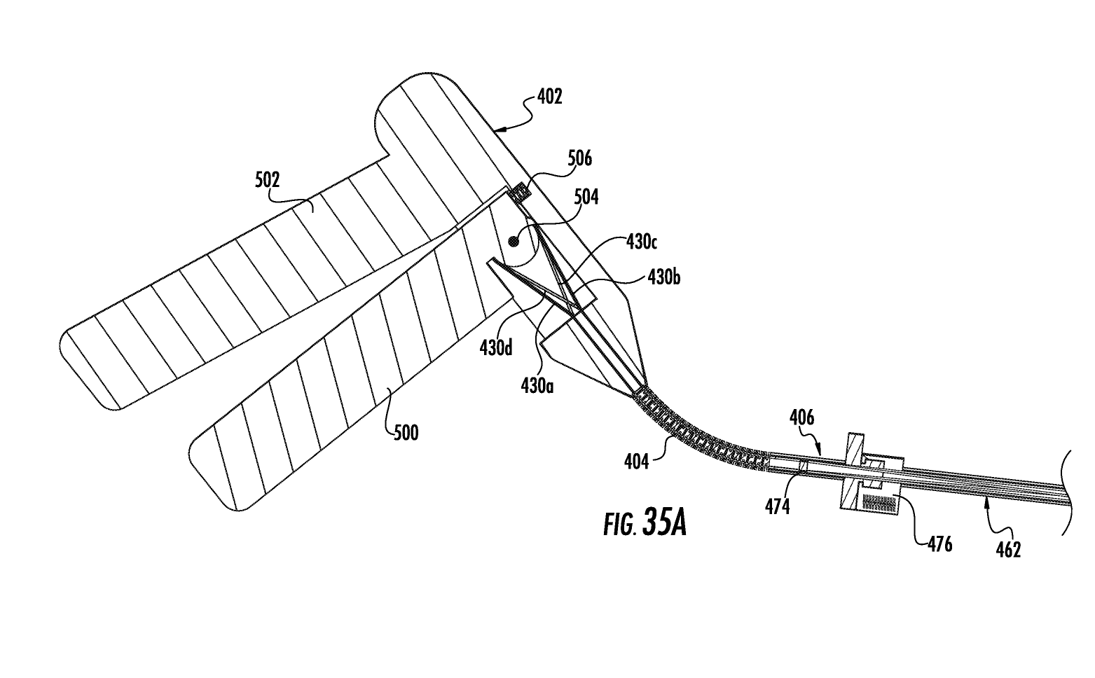

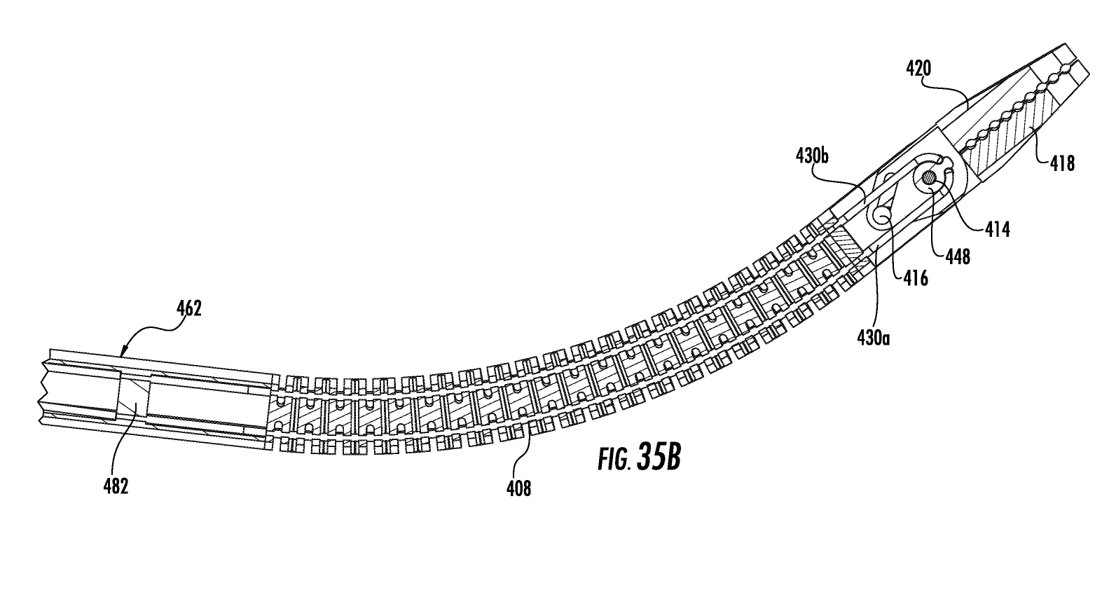

FIGS. 36A and 36B are views that compress the distance between the three cable guides 474, 482, 484 and with the tube assembly 406 removed for the sake of illustration in order to show how the cable configuration is altered by changing the orientation of the distal tube element 462, and how this causes a control mode change. Each cable guide 474, 482, 484 may be essentially a disk with four holes to guide the four control cables 430a, 430b, 430c, 430d. The proximal, first cable guide 474, positioned near the proximal articulation element 404, is in a fixed orientation within the proximal tube element 460, which also is fixed relative to the manipulator 402, and thus the orientation of the cables 430a, 430b, 430c, 430d is fixed at this location. The second cable guide 484 is positioned approximately at the middle of the distal tube element 462. The third cable guide 482 is positioned proximate to the distal end of the distal tube element 462. The second and third cable guides 482, 484 are in a fixed orientation within the distal tube element 462, but the distal tube element, as previously discussed, is rotatable relative to the proximal tube element 40.

FIG. 36A shows a motion-following control mode. From the user's perspective, cable 430a enters the first cable guide 474 at the bottom right position and enters the second cable guide 484 in the distal tube element 462 at the top right position. Cable 430b changes from the top right position to the top left position, cable 430d changes from the top left position to the bottom left position, and cable 430c changes from the bottom left to the bottom right position. The cables may be considered to have shifted 90 degrees counterclockwise. After exiting the second cable guide 484, the cables 430a, 430b, 430c, 430d enter the third cable guide 482, also in the distal tube element 462. The changes in position between the second cable guide 484 and the third cable guide 482 are reversed such that the positions of the cables 430a, 430b, 430c, 430d in the first cable guide 474 and third cable guide 482 are the same, with the cables 430a, 430b, 430c, 430d having shifted 90 degrees clockwise. After exiting the third cable guide 482, the cables 430a, 430b, 430c, 430d enter the distal articulation element 408.

In FIG. 36B, the distal tube element 462 and distal end of the instrument 400 have been rotated 180 degrees about the longitudinal axis of the tube assembly 406 by a mode change to yield a motion-mirrored control mode. Since the end effector 410 is symmetric about this axis, the mode change does not affect the end effector's operation. The entire assembly, from the distal tube element 462 onward distally, has been rotated 180 degrees. This twists the cables between the first cable guide 474 and second cable guide 484 into a new configuration. Within the distal tube element 462 the cabling is unchanged, though the cabling orientation from an external perspective has been rotated 180 degrees. In the first cable guide 474, from the perspective of the user and, as in FIG. 39 since the first cable guide 474 does not move, cable 430a is in the bottom right position, cable 430b is in the top right position, cable 430c is in the bottom left position, and cable 430d is in the top left position. Between the first cable guide 474 and the second cable guide 484, cable 430a moves to the bottom left position, cable 430b moves to the bottom right position, cable 430c moves to the top left position, and cable 430d moves to the top left position. Between the second cable guide 484 and third cable guide 482, cable 430a moves to the top left position, cable 430b moves to the bottom left position, cable 430c moves to the top right position, and cable 430d moves to the bottom right position.

The length of the control cables between the first and second cable guides 474, 482 cable guides is the same in either mode, and as such the tension in those cables and their response to the actions of the proximal articulation element 404 and manipulator 402 will not be affected by changing control modes. When the manipulator 402 is moved upward, cables 430a and 430c are retracted, which causes an upward deflection of the distal articulation element 408 in this configuration. Similar behavior will be caused by downward and lateral deflections of the manipulator 402. In this manner, the end effector 410 is controlled in the same way as in the motion-following control mode, but the response of the distal articulation element 408 is the opposite of its response in the motion-following control mode. This provides an alternate means of controlling the distal portion of the instrument 400 for those who prefer this style of control.

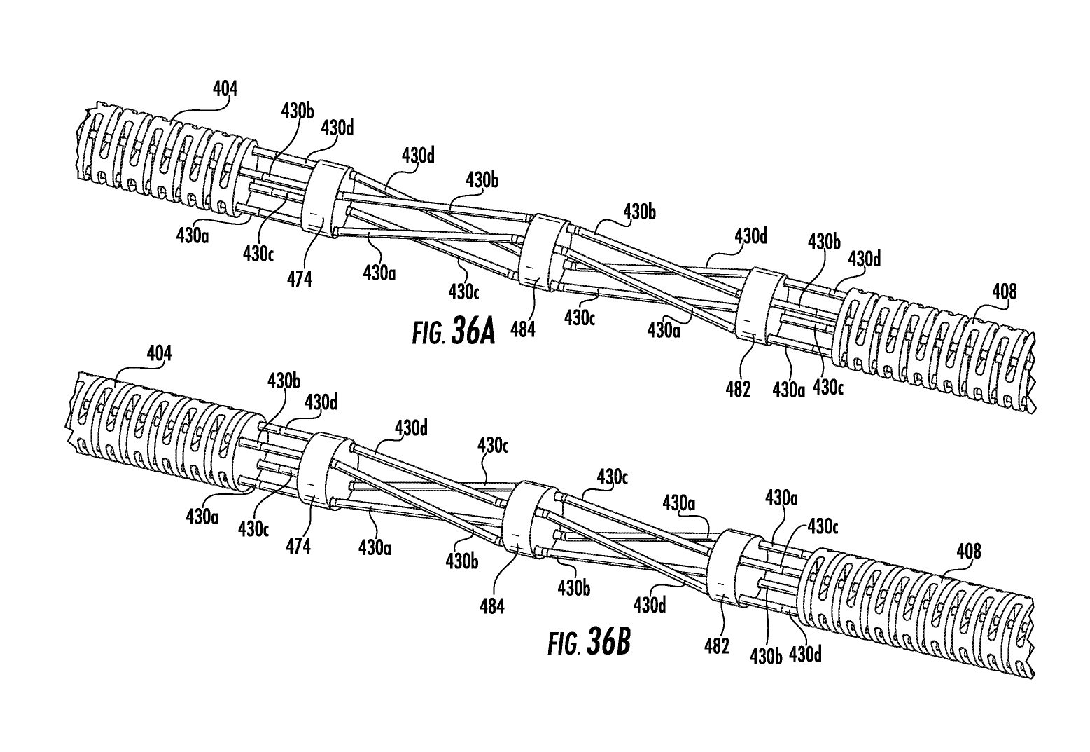

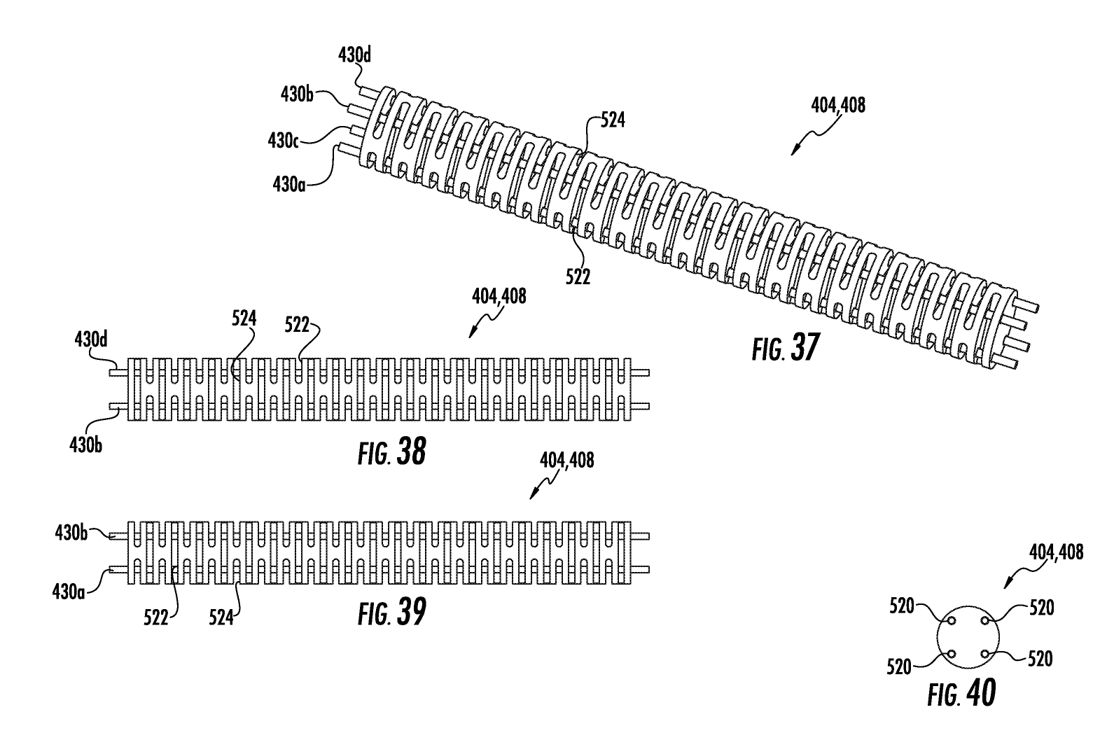

FIGS. 37-40 show the articulation elements, with cabling included in FIGS. 37-39. The proximal and distal articulation elements 404, 408 may be essentially identical, and therefore only one element is shown as a representation of both. In this instance, for example, the elements may be fabricated from plastic, such as plastic rods with an array of four holes 520 bored longitudinally to allow the passage of four control cables 430a, 430b, 430c, 430d, and alternating vertical cuts 522 and horizontal cuts 524 to omit material to increase the flexibility of the elements. Material may be omitted in manners other than by performing cuts. When two adjacent cables are retracted, the articulation element is bent in the direction of those cables. For example, retracting cables 430a and 430b or retracting cables 430c and 430d will cause a deflection in the horizontal plane, whereas retracting cables 430a and 430c or cable 430b and 430d will cause a deflection in the vertical plane. Other deflections can be caused by combinations of these actions.

While the materials of the instrument are not intended to be constrained, the material for many of the parts may be expected to be surgical grade, including stainless steel or plastic, or other materials as known to one of ordinary skill in the art. Universal joints, jaw assemblies, and tubes may be made of stainless steel. The manipulator may be made of hard plastic and metal components. As noted above, the flexible articulation elements may be made, in one embodiment, of flexible plastic with alternating partial horizontal and vertical cuts. Cables may be made of, for example, stainless steel rope, aramid fiber cables, or aligned fiber cables. Other materials may be selected as known to one of ordinary skill in the art. Dimensions may be selected based on the application. Conventional diameters, which may apply to embodiments described herein, include tube, distal joint, end effector, and end segment diameters of 5 or 10 mm, or as appropriate for the cannula through which the instrument must pass.

The surgical instrument may include the characteristic of interchangeability of components. For example, the manipulators and end effectors other than those previously described may be provided. Further, in some embodiments manually operated manipulators may be replaced by robotic manipulators.

Although only a few exemplary embodiments have been shown and described in considerable detail herein, it should be understood by those skilled in the art that it is not intended to be limited to such embodiments since various modifications, omissions and additions may be made to the disclosed embodiments without materially departing from the novel teachings and advantages, particularly in light of the foregoing teachings. Accordingly, it is intended to cover all such modifications, omission, additions and equivalents as may be included within the spirit and scope as defined by the following claims. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

1-38. (canceled)

39. A surgical tool for use by an operator, comprising:
a proximal joint that constrains pivoting of parts mounted and adjacent to the proximal joint to be about two perpendicular, intersecting axes including a primary axis and a secondary axis, wherein the proximal joint has two degrees of freedom, the proximal joint including a first end and a second end;
a hollow elongated member having a longitudinal axis and including a first end and a second end, the elongated member first end being mounted to the proximal joint second end;
a distal joint that constrains pivoting of parts mounted and adjacent to the distal joint to be about two perpendicular, intersecting axes including a primary axis and a secondary axis, wherein the distal joint has two degrees of freedom, the distal joint including a first end and a second end, and the distal joint first end being mounted to the elongated member second end; and
three articulation control cable lengths comprising a first cable, a second cable, and a third cable that engage and operatively couple the proximal joint and distal joint to control the two degrees of freedom of the distal joint.

40. The surgical tool of claim 39, wherein the proximal joint is a universal joint and the distal joint is a universal joint.

41. The surgical tool of claim 39, wherein the proximal joint includes a proximal yoke at the first end, a distal yoke at the second end, a center block between the proximal joint proximal yoke and proximal joint distal yoke, and means for mounting the proximal joint proximal yoke and the proximal joint distal yoke to the proximal joint center block that permit pivoting the proximal joint proximal yoke and proximal joint distal yoke about the two perpendicular, intersecting axes through the proximal joint center block, and
wherein the distal joint includes a proximal yoke at the first end, a distal yoke at the second end, a center block between the distal joint proximal yoke and distal joint distal yoke, and means for mounting the distal joint proximal yoke and the distal joint distal yoke to the distal joint center block that permit pivoting the distal joint proximal yoke and distal joint distal yoke about the two perpendicular, intersecting axes through the distal joint center block.

42. The surgical tool of claim 41, wherein the first cable and the second cable are attached to the proximal yoke of the proximal joint and to the distal yoke of the distal joint.

43. The surgical tool of claim 42, wherein the third cable is attached to the proximal joint center block and the distal joint center block.

44. The surgical tool of claim 41, wherein each yoke has a first arm and a second arm that oppose each other,
wherein the first cable is attached to an arm of the proximal yoke of the proximal joint and to an arm of the distal yoke of the distal joint,
wherein the second cable is attached to an arm of the proximal yoke of the proximal joint and to an arm of the distal yoke of the distal joint,
wherein the third cable is attached to the center block of the proximal joint and to the center block of the distal joint, and
wherein pivoting the proximal yoke of the proximal joint causes a corresponding motion of the distal yoke of the distal joint.

45. The surgical tool of claim 44, wherein pivoting the proximal yoke of the proximal joint in a predetermined direction causes retraction of the first and second cables distally and relaxing of the third cable.

46. The surgical tool of claim 44, wherein the first cable and second cable follow a path partially around each center block in a first direction and the third cable follows a path partially around each center block in a second, opposite direction.

47. The surgical tool of claim 46, wherein each center block defines a groove for receiving the third cable.

48. The surgical tool of claim 47, wherein each center block is cylindrical with a portion having a greater radius than the remainder of the center block.

49. The surgical tool of claim 48, wherein the portion having a greater radius forms a proximal face and a distal face.

50. The surgical tool of claim 49, wherein the proximal face and the distal face are offset by approximately one quarter of the circumference of the center block.

51. The surgical tool of claim 50, wherein the groove is defined by the portion having a larger radius and passes through the portion having a larger radius along the circumference of the center block.

52. The surgical tool of claim 46, wherein the distal yoke of the proximal joint and the proximal yoke of the distal joint each comprise cable guides, each cable guide comprising a curved surface.

53. The surgical tool of claim 52, wherein the first cable and the second cable each engage a cable guide on the distal yoke of the proximal joint, the center block of the proximal joint, and a round feature on an arm of the proximal yoke of the proximal joint, and wherein the first cable and the second cable each engage a cable guide on the proximal yoke of the distal joint, the center block of the distal joint, and a round feature on an arm of the distal yoke of the distal joint.

54. The surgical tool of claim 39, wherein the three cable lengths terminate in the proximal joint and the distal joint.

55. The surgical tool of claim 39, further comprising:
a manipulator adapted to receive at least a portion of the operator's hand, the manipulator mounted to the proximal joint first end;
an end effector mounted to the distal joint second end; and
one end effector actuation cable that engages and operatively couples the manipulator and the end effector.

56. The surgical tool of claim 55, wherein pivoting the manipulator about the primary axis of the proximal joint in a predetermined first direction at a first angle from the longitudinal axis causes the first cable and the second cable to retract and the third cable to relax, and causes the distal yoke of the distal joint to pivot in a second direction about the primary axis of the distal joint at the same angle from the longitudinal axis and on the opposite side of the longitudinal axis as the first angle.

57. The surgical tool of claim 55, wherein the end effector comprises two movable jaws that operate simultaneously.

58. The surgical tool of claim 57, wherein the end effector further comprises a jaw actuation pin to which the end effector actuation cable is attached, wherein the jaw actuation pin is received in a slot in each jaw and proximal movement of the jaw actuation pin causes the two movable jaws to close.

59. A surgical tool for use by an operator, comprising:
a proximal flexible articulation element including a first end and a second end;
a tube assembly having an longitudinal axis and comprising a proximal tube element and a distal tube element, the proximal tube element including a proximal end mounted to the second end of the proximal flexible articulation element and a distal end, the distal tube element including a distal end and a proximal end rotatably mounted to the distal end of the proximal tube element such that the distal tube element may rotate about the longitudinal axis relative to the proximal tube element; and
a distal flexible articulation element including a first end and a second end, the first end of the distal flexible articulation element being mounted to the distal end of the distal tube element.

60. The surgical tool of claim 59, further comprising:
a manipulator adapted to receive at least a portion of the operator's hand, the manipulator being mounted to the first end of the proximal flexible articulation element;
an end effector including at least one movable jaw, the end effector mounted to the second end of the distal flexible articulation element; and
cables that engage and operatively couple the manipulator, proximal flexible articulation element, and distal flexible articulation element and that concurrently engage and operatively couple the manipulator and the end effector.

61. The surgical tool of claim 60, wherein when the distal tube element is in a first angular orientation with respect to the proximal tube element, the surgical tool is in a motion-following mode, and when the distal tube element is in a second angular orientation with respect to the proximal tube element, the surgical tool is in a motion-mirroring mode.

62. The surgical tool of claim 60, wherein, in a first mode of operation, pivoting the manipulator and the proximal flexible articulation element in a predetermined first direction at a first angle from the longitudinal axis causes the distal flexible articulation element to pivot in a second direction at the same angle from the longitudinal axis and on the opposite side of the longitudinal axis as the first angle, and wherein, in a second mode of operation, pivoting the manipulator and the proximal flexible articulation element in the predetermined first direction at the first angle from the longitudinal axis causes the distal flexible articulation element to pivot in a third direction at the same angle from the longitudinal axis and on the same side of the longitudinal axis as the first angle.

63. The surgical tool of claim 59, wherein the proximal tube element comprises a proximal tube including a first rotational mounting means mounted proximate to the distal end of the proximal tube element, and wherein the distal tube element comprises a distal tube including a second rotational mounting means mounted to the first rotational mounting means.

64. The surgical tool of claim 63, wherein the first rotational mounting means comprises a flange and an annulus spaced from the flange along the longitudinal axis, and the second rotational mounting means comprises a lever including a collar that is longitudinally secured and rotationally mounted to the annulus.

65. The surgical tool of claim 64, wherein the flange includes a distal surface that defines depressions offset by 180 degrees around the longitudinal axis, and further comprising a retention means mounted to the distal tube element that engages the depressions to maintain the rotational position of the distal tube element relative to the proximal tube element.

66. The surgical tool of claim 65, wherein the retention means is a locking spring plunger.

67. The surgical tool of claim 60, further comprising a first cable guide disposed in the proximal tube, a second cable guide disposed in the distal tube proximate to the end effector, and a third cable guide disposed in the distal tube between the first cable guide and the second cable guide.

68. The surgical tool of claim 67, wherein each of the cable guides define four holes parallel to and evenly distributed about the longitudinal axis to receive the cables.

69. The surgical tool of claim 68, wherein the first cable guide is in a fixed angular position relative to the proximal tube and the manipulator, and the second cable guide and third cable guide are each in a fixed angular position relative to the distal tube and each other.

70. The surgical tool of claim 69, wherein when the distal tube element is rotated about the longitudinal axis from the first angular orientation by 180 degrees to the second angular orientation, the position of the holes and the cables extending distally from the third cable guide in the distal tube is shifted about the longitudinal axis by 180 degrees, and the end effector rotates about the longitudinal axis 180 degrees.

71. The surgical tool of claim 60, wherein the cables comprise four cable lengths that control both the deflection of the distal flexible articulation element and the operation of the at least one movable jaw.

72. The surgical tool of claim 71, wherein the four cable lengths comprise two cables terminating in the manipulator and fixed to the end effector.

73. The surgical tool of claim 71, wherein the four cable lengths comprise four separate cables, each terminating in the manipulator and fixed to the end effector.

74. The surgical tool of claim 60, wherein the at least one movable jaw comprises two movable jaws that operate simultaneously.

75. A method of operating a surgical tool, the surgical tool comprising a manipulator adapted to receive at least a portion of the operator's hand, a proximal flexible articulation element including a first end and a second end, the first end of the proximal flexible articulation element being mounted to the manipulator, a tube assembly having an longitudinal axis and comprising a proximal tube element and a distal tube element, the proximal tube element including a proximal end mounted to the second end of the proximal flexible articulation element and a distal end, the distal tube element including a distal end and a proximal end rotatably mounted to the distal end of the proximal tube element such that the distal tube element may rotate about the longitudinal axis relative to the proximal tube element, a distal flexible articulation element including a first end and a second end, the first end of the distal flexible articulation element being mounted to the distal end of the distal tube element, an end effector including at least one movable jaw, the end effector mounted to the second end of the distal flexible articulation element, and cables that engage and operatively couple the manipulator, proximal flexible articulation element, and distal flexible articulation element and that concurrently engage and operatively couple the manipulator and the end effector, the method comprising:
pivoting the manipulator and the proximal flexible articulation element in a predetermined first direction at a first angle from the longitudinal axis to cause the distal flexible articulation element to pivot in a second direction at the same angle from the longitudinal axis and on the opposite side of the longitudinal axis as the first angle;
changing the mode of operation of the surgical tool; and
pivoting the manipulator and the proximal flexible articulation element in the predetermined first direction at the first angle from the longitudinal axis to cause the distal flexible articulation element to pivot in a third direction at the same angle from the longitudinal axis and on the same side of the longitudinal axis as the first angle.

76. The method of claim 75, wherein changing the mode of operation of the surgical tool comprises rotating the distal tube element about the longitudinal axis 180 degrees relative to the proximal tube element.
